SEQUENCE LISTING (19 sequences)

SEQ ID NO: 1 - protein, 12 aa, synthetic construct
EEWXXWXPPFFX

SEQ ID NO: 2 - protein, 12 aa, synthetic construct
ENFWWWNWRXWG

SEQ ID NO: 3 - protein, 12 aa, synthetic construct
FRWNFHRNTFFP

SEQ ID NO: 4 - protein, 12 aa, synthetic construct
GARRENXFFAAF

SEQ ID NO: 5 - protein, 12 aa, synthetic construct
HPRRTHRWWWWE

SEQ ID NO: 6 - protein, 12 aa, synthetic construct
HPHEAHRPPHRH

SEQ ID NO: 7 - protein, 12 aa, synthetic construct
IRHHNIWFNTIA

SEQ ID NO: 8 - protein, 12 aa, synthetic construct
IIHRRAFWWFPN

SEQ ID NO: 9 - protein, 12 aa, synthetic construct
NNWWRHARIINA

SEQ ID NO: 10 - protein, 12 aa, synthetic construct
PRNWRPIHAIIH

SEQ ID NO: 11 - protein, 12 aa, synthetic construct
RAREHHWWREEH

SEQ ID NO: 12 - protein, 12 aa, synthetic construct
RERNRHFPHFTH

SEQ ID NO: 13 - protein, 12 aa, synthetic construct
RWTIWPPNWHPP

SEQ ID NO: 14 - protein, 12 aa, synthetic construct
RWPRTHHNPAAA

SEQ ID NO: 15 - protein, 12 aa, synthetic construct
RITRTFNPPPFW

SEQ ID NO: 16 - protein, 12 aa, synthetic construct
TRTRHFRPPEEE

SEQ ID NO: 17 - protein, 12 aa, synthetic construct
TPTFRWHNFFPI

SEQ ID NO: 18 - protein, 12 aa, synthetic construct
TFAATFNPAHNA

SEQ ID NO: 19 - protein, 12 aa, synthetic construct
WNPIPSYYPFFR

(19) United States
(12) Patent Application Publication
(10) Pub. No.: US 2026/0062442 A1
(43) Pub. Date: Mar. 5, 2026

(54) COMPOSITIONS AND METHODS FOR PURIFYING ANTIGEN-BINDING ANTIBODY FRAGMENTS USING PEPTIDE LIGANDS

(71) Applicant: North Carolina State University, Raleigh, NC (US)

(72) Inventors: Stefano Menegatti, Raleigh, NC (US); Ryan E. Kilgore, Raleigh, NC (US); Wenning Chu, Raleigh, NC (US)

(21) Appl. No.: 19/118,176

(22) PCT Filed: Oct. 4, 2023
(86) PCT No.: PCT/US2023/075984

§ 371 Date: Apr. 3, 2025

Related U.S. Application Data
(60) Provisional application No. 63/378,302, filed on Oct. 4, 2022.

(51) Int. Cl. C07K 4/00; C07K 1/04; C07K 1/22; C07K 16/00; C07K 17/02; C07K 17/14

(52) U.S. Cl. CPC C07K 4/00; C07K 1/04; C07K 1/22; C07K 16/00; C07K 17/02; C07K 17/14; C07K 2317/24; C07K 2317/31; C07K 2317/55; C07K 2317/622; C07K 2319/00

(57) ABSTRACT

The present disclosure provides compositions and methods related to the purification and/or isolation of antibodies. In particular, the present disclosure provides novel peptide ligands capable of targeting the fragment antigen binding (Fab) domain and/or single-chain variable fragment (scFv) of antibodies to facilitate the isolation and/or purification of antibodies from processing fluid streams. The novel ligands disclosed herein are capable of universal and subtype-specific biorecognition.

### CROSS REFERENCE TO RELATED APPLICATIONS

This application claims priority to and the benefit of U.S. Provisional Patent Application No. 63/378,302 filed Oct. 4, 2022, which is incorporated herein by reference in its entirety and for all purposes.

### SEQUENCE LISTING STATEMENT

The contents of the electronic sequence listing titled (NCSU-41281-601.xml; Size: 17,561 bytes; and Date of Creation: Oct. 3, 2023) is herein incorporated by reference in its entirety.

### FIELD

The present disclosure provides compositions and methods related to the purification and/or isolation of antibodies. In particular, the present disclosure provides novel peptide ligands capable of targeting the fragment antigen binding (Fab) domain and/or single-chain variable fragment (scFv) to facilitate the isolation and/or purification of Fab- and/or scFv-containing proteins or polypeptides (e.g., antibodies) from processing fluid streams. The novel ligands disclosed herein are capable of universal and subtype-specific biorecognition.

### BACKGROUND

Recent developments in engineering monoclonal antibodies (mAbs) have greatly increased the therapeutic arsenal for fighting cancer, autoimmune and degenerative disorders, and infections. Of particular interest are the classes of multispecific monoclonal antibodies (msAbs) and antibody domains and/or fragments, which are designed to improve targeting precision and strength, or promote the recruitment of T-Cells and natural killer cells to the disease site; furthermore, antibody domains and/or fragments feature better tissue penetration and reduced immunogenicity compared to full mAbs, while featuring comparable targeting selectivity. While to date only three therapeutic Fabs have been approved by the FDA—i.e., abciximab, ranibizumab, and certolizumab-hundreds of products featuring the single-domain or multi-specific format have accessed clinical trials worldwide and many more are in preclinical research.

While displaying superior therapeutic efficacy compared to conventional mAbs, these products mandate more complex biomanufacturing processes, being often expressed at low titer or more propense to aggregate or more challenging to purify. Unlike conventional mAbs, in fact, multispecific full antibodies comprise two or more Fab domains with different biomolecular features (K and λ types); similarly, antibody domains and/or fragments comprise multiple antigen-targeting segments, but are devoid of the Fc domain. As a result, the current Protein A-based purification platform is either insufficient or altogether inadequate to purify msAbs, which requires multiple, orthogonal steps of affinity chromatography targeting the Fc, Fab-κ, and Fab-λ. The Fab-binding counterpart of Protein A—the Finegoldia magna's Protein L—binds only Fab-κ1, κ3, and κ4, but does not bind to Fab-κ2 or any of the Fab-λ subtypes, and is therefore unable to isolate κ/λ-msAbs. These limitations have spurred considerable efforts toward developing novel ligands targeting Fab-κ and Fab-λ, aimed at establishing a downstream process pipeline for msAbs. In this context, engineered camelid antibody-based ligands KappaSelect and LambdaFabSelect were introduced in the early 2010s. Like Protein A, however, these ligands impose the use of harsh elution conditions, are biochemically labile and can release immunogenic fragments in the product stream, and are very expensive (up to $18,000 per liter of resin).

Peptide ligands provide significant advantages over conventional protein ligands: besides their excellent biorecognition activity, peptides feature stronger biochemical stability and safety, and can be manufactured at scale more affordably, thus significantly reducing the cost of affinity resins. Furthermore, a diverse toolbox is now available for engineering peptides targeting any desired product with controlled affinity: this is a particularly desirable characteristic, as it enables mild elution, thus reducing the risk of compromising product stability or triggering ligand fragmentation and leaching.

### SUMMARY

Embodiments of the present disclosure include novel peptide ligands capable of targeting the fragment antigen binding (Fab) domain and/or single-chain variable fragment (scFv) to facilitate the isolation and/or purification of Fab- and/or scFv-containing proteins or polypeptides (e.g., antibodies) from processing fluid streams. In accordance with these embodiments, the present disclosure provides a peptide ligand (or a plurality of peptide ligands) comprising no more than 12 amino acids and having the amino acid sequence X₁X₂X₃X₄X₅X₆X₇X₈X₉X₁₀X₁₁X₁₂, wherein: (i) positions X₁-X₅ comprise at least one positively charged amino acid or an aromatic amino acid; (ii) positions X₆-X₈ comprise at least one aromatic amino acid; and (iii) positions X₈-X₁₂ comprise at least one aliphatic amino acid or at least one aromatic amino acid. In some embodiments, the peptide ligand is capable of binding a target fragment antigen binding (Fab) domain of a Fab-containing protein or polypeptide. In some embodiments, the peptide ligand is capable of binding a single-chain variable fragment (scFv) of a scFv-containing protein or polypeptide.

In some embodiments, the at least one peptide ligand comprises at least one histidine or arginine residue.

In some embodiments, the peptide ligand is capable of binding a target Fab comprising a lambda (λ) light chain isotype. In some embodiments, the peptide ligand is capable of binding a target Fab comprising a kappa (κ) light chain isotype. In some embodiments, the peptide ligand is capable of binding a target Fab comprising a lambda (λ) light chain isotype and a target Fab comprising a kappa (κ) light chain isotype. In some embodiments, the Fab domain comprises a single-chain variable fragment (scFv), and the peptide ligand is capable of binding the scFv.

In some embodiments, the Fab and/or the scFv is comprised within a monoclonal antibody or a polyclonal antibody. In some embodiments, the Fab and/or the scFv is comprised within a mono-specific antibody or a multi-specific antibody. In some embodiments, the Fab and/or the scFv is comprised within a single-domain antibody, a humanized antibody, or a chimeric antibody. In some embodiments, the Fab and/or the scFv is comprised within a fusion protein.

In some embodiments, the peptide ligand comprises one or more of alanine (A), glutamic acid (E), phenylalanine (F), histidine (H), isoleucine (I), asparagine (N), proline (P), arginine (R), threonine (T), and tryptophan (W).

In some embodiments, the peptide ligand exhibits a maximum equilibrium binding capacity (Qₘₐₓ) of at least about 15 mg of Fab/mL resin. In some embodiments, the peptide ligand exhibits a disassociation constant (K_{D}) less than or equal to about 5×10⁻⁶ M. In some embodiments, the peptide ligand exhibits a dynamic binding capacity (DBC_{10%}) from about 5 mg/mL to about 20 mg/mL.

In some embodiments, the peptide ligand comprises an amino acid sequence having at least 90% sequence identity with any of SEQ ID NOs: 1-19. In some embodiments, the peptide ligand comprises an amino acid sequence having at least 90% sequence identity with SEQ ID NO: 3.

In some embodiments, the peptide ligand comprises a linker, and wherein the linker is capable of binding to a solid support. In some embodiments, the linker is bound to the C-terminus of the peptide ligand, and wherein the linker comprises a Glyn or a [Gly-Ser-Gly]ₘ, wherein 10≥n≥1 and 6≥m≥1. In some embodiments, the peptide ligand is bound to a solid support. In some embodiments, the solid support comprises a non-porous or porous particle, a membrane, a polymer surface, a fiber or a woven or non-woven fibermat, a hydrogel, a microplate, and/or a microfluidic device. In some embodiments, the porous particle is a chromatograph resin. In some embodiments, the solid support comprises polyacrylate, polyacrylamide, poly-ether, polyolefin, polyester, polysaccharide, iron oxide, silica, titania, and/or zirconia.

Embodiments of the present disclosure also include a composition for purifying a target biologic (e.g., a Fab-containing protein or polypeptide and/or a scFv-containing protein or polypeptide) from a fluid using any of the peptide ligands described herein.

In some embodiments, the fluid is a cell culture fluid. In some embodiments, the fluid comprises a supernatant and/or a cellular lysate. In some embodiments, the fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof. In some embodiments, the biological fluid is derived from HEK293 cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the biological fluid is derived from yeast cells or fungal cells. In some embodiments, the yeast cells are Pichia pastoris cells.

In some embodiments, the fluid comprises a pH from about 3.0 to about 9.0.

In some embodiments, the target biologic is: (i) a monoclonal antibody or a polyclonal antibody; (ii) a mono-specific antibody or a multi-specific antibody; (iii) a single-domain antibody, a humanized antibody, or a chimeric antibody; or (iv) a Fab domain, a scFv fragment, or fusion proteins comprising Fab or scFv.

Embodiments of the present disclosure also include an adsorbent for purifying a target biologic (e.g., a Fab-containing protein or polypeptide and/or a scFv-containing protein or polypeptide) from a fluid using any of the peptide ligands described herein.

Embodiments of the present disclosure also include a method of purifying a target biologic from a fluid. In accordance with these embodiments, the method includes contacting the fluid with any of the peptide ligands described herein, or any of the adsorbents described herein, under conditions sufficient for the peptide ligands or adsorbents to bind the target biologic; and eluting the target biologic from the peptide ligands.

In some embodiments, the conditions sufficient for the peptide ligands to bind the target biologic comprise use of a buffer comprising: (i) 10-100 mM phosphate buffer with 0-1 M NaCl at pH 6.5-8.5; or (ii) 10-100 mM Tris buffer with 0-1 M NaCl at pH 6.5-8.5. In some embodiments, the conditions sufficient for the peptide ligands to bind the target biologic comprise use of a buffer comprising a pH ranging from about pH 6.0 to about pH 9.0.

In some embodiments, eluting the target biologic from the peptide ligands comprises use of a buffer comprising: (i) 0.1 M glycine pH 2.5-3.6; or (ii) 0.2 M acetate buffer pH 3.6-5. In some embodiments, eluting the target biologic from the peptide ligands comprises use of a buffer comprising a pH ranging from about pH 2.0 to about pH 5.0.

In some embodiments, the method results in a yield for the target biologic of at least 70%. In some embodiments, the method results in a purity for the target biologic of at least 70%.

### BRIEF DESCRIPTION OF THE DRAWINGS

FIGS. 1A-1B: Selection of Fab-binding peptides. (A) A library of 12-mer peptide-ChemMatrix beads was (1) incubated with a screening feed comprising AF594-labeled Fab-κ and AF488-labeled CHO host cell proteins, washed, and (2) fed to the bead-sorting microfluidic device; (3) green-only and red-and-green beads were discarded, whereas the red-only beads were selected as positive; (4) the red-only beads were exposed to model elution conditions (0.2 M acetate buffer, pH 4); (5) the beads that displayed a loss of red fluorescence >20% were analyzed by Edman degradation to identify candidate Fab-binding sequences. (B) Weblogo plot of the selected sequences.

FIG. 2: Chromatograms obtained by loading a solution of human polyclonal Fab at 2 mg/mL in PBS on a 0.1 mL column packed with IRHHNIWFNTIA-, NNWWRHARIINA-, RWTIWPPNWHPP-, IIHRRAFWWFPN-, and FRWNFHRNTFFP-Toyopearl resin at the residence time of 2 min; Fab elution was performed at pH 4.

FIGS. 3A-3B: Chromatograms obtained by loading (A) Fab-κ and (B) Fab-λ at 1 mg/mL in PBS on a 0.1 mL column packed with IRHHNIWFNTIA-, RWTIWPPNWHPP-, NNWWRHARIINA-, IIHRRAFWWFPN-, or FRWNFHRNTFFP-Toyopearl resin at the residence time of 2 min; Fab elution was performed at pH 4; the effluents were continuously monitored via spectrophotometry at 280 nm.

FIG. 4: Breakthrough curves of human Fab loaded on FRWNFHRNTFFP-Toyopearl resin at different concentration namely, 1, 2, and 5 mg/mL and residence time (RT) of either 1, 2, or 5 min; Protein L-based resins loaded with a 1 mg/mL solution of Fab in PBS at the RT of either 1 or 2 min were used as controls.

FIG. 5: Chromatograms obtained by loading human Fab at 2 mg/mL in PBS on a 0.1 mL column packed with FRWNFHRNTFFP-Toyopearl, WIPNSEFEHERTK-Toyopearl (B1), HQNHHSTFRWEIY-Toyopearl (C7), and WHYNWQDVSDRQ-Toyopearl (A5) resins at the residence time of 2 min; Fab elution was performed at pH 4; the effluents were continuously monitored via spectrophotometry at 280 nm.

FIGS. 6A-6E: In silico binding of (A) FRWNFHRNTFFP-GSG (red), (B) IIHRRAFWWFPN-GSG (blue), (C) IRHHNIWFNTIA-GSG (green), (D) NNWWRHARIINA-GSG (salmon), and (E) RWTIWPPNWHPP-GSG (yellow) peptides on the homology structures of Fab-κ and Fab-λ. The Fab-binding segment of the peptides is in colored cartoon, while the GSG spacer is in grey cartoon; the V_{H} and C_{H}1 domains of Fab-κ and Fab-λ are in blue-white and light blue cartoons, respectively; the V_{L} and C_{L} domains of Fab-κ and Fab-λ are in pink and light orange cartoons, respectively; Protein L is in light pink cartoon; the putative binding site 1 (green), site 2 (yellow-orange), site 3 (wheat), site 4 (blue), site 5 (sand), site 6 (pink), site 7 (yellow), and site 8 (orange) are in colored mesh.

FIGS. 7A-7B: Equilibrium binding isotherms of Fab and Fab-k on (A) FRWNFHRNTFFP-Toyopearl resin, (B) IIHRRAFWWFPN-Toyopearl resin, and (C) Protein L-Sepharose resin.

FIGS. 8A-8B: SDS-Page (non-reducing conditions) of the (A) flow-through and (B) elution fractions generated by purifying Fab-κ from an industrial CHO cell culture harvest using FRWNFHRNTFFP-Toyopearl resin. Protein binding was conducted at different values of NaCl concentration (i.e., 20 mM, 150 mM, and 500 mM) and pH (i.e., 6.5, 7.5, and 8.5), while elution was performed using 0.2 M acetate buffer at pH 4.

FIGS. 9A-9D: (A) SDS-PAGE analysis (non-reducing conditions, silver stained) of human Fab, null CHO-S CCCF, the feedstock comprising human polyclonal Fab at 1 mg/mL in CHO-S cell culture fluid with an HCP titer of 0.135 mg/mL, and the flow-through (FT) and elution (El) fractions generated by processing the model feedstock using FRWNFHRNTFFP-Toyopearl and Protein L-agarose resins. SEC analysis of (B) the model feedstock (black), the null CHO-S cell culture fluid with an HCP titer of 0.135 mg/mL (purple), and pure Fab (yellow); the flow-through (red) and elution (blue) fractions obtained by injecting 0.1 mL of feedstock in a 0.1 mL column packed with (C) FRWNFHRNTFFP-Toyopearl resin or (D) Protein L-agarose resin; the trace obtained with the pure elution buffer is in green.

FIGS. 10A-10C: SEC analysis of (A) the model feedstock comprising Certolizumab Fab at 1 mg/mL in a null CHO-S cell culture fluid with an HCP titer of 0.109 mg/mL (black) and pure Fab (yellow); (B) the flow-through (red) and elution (blue) fractions obtained by injecting 0.1 mL of feedstock in a 0.1 mL column packed with FRWNFHRNTFFP-Toyopearl resin; the trace obtained with the pure elution buffer is in green. (C) SDS-PAGE analysis (non-reducing conditions) of Certolizumab, the model feedstock, and the flow-through (FT), elution (E), and regeneration (R) fractions generated by processing the model feedstock using FRWNFHRNTFFP-Toyopearl resin.

FIGS. 11A-11D: (A) SDS-PAGE (non-reducing conditions) of the model feedstock comprising a therapeutic monoclonal Fab at 0.171 mg/mL in a clarified CHO-K1 cell culture harvest with a HCP titer of 0.157 mg/mL, and the flow-through (FT) and elution (El) fractions generated by purifying Fab from the model feedstock using FRWNFHRNTFFP-Toyopearl and Protein L-agarose resins; the adsorption was conducted at 20 mM NaCl concentration and pH 7.5; elution for FRWNFHRNTFFP-Toyopearl was performed using 0.2 M acetate buffer at pH 4 and elution for Protein L-agarose was performed using 0.1 M glycine buffer at pH 2.5. SEC analysis of (B) the model feedstock, and the flow-through (red) and elution (blue) fractions obtained by injecting 0.5 mL of feedstock in a 0.1 mL column packed with (C) FRWNFHRNTFFP-Toyopearl resin or (D) Protein L-agarose resin; the trace obtained with the pure elution buffer is in green.

FIGS. 12A-12B: (A) Comparison of 20 cycles of purification of human Fab from a CHO CCCF using FRWNFHRNTFFP-Toyopearl; labels: FT, flowthrough fraction; El, elution; R, regeneration. (B) Values of relative Fab yield and purity obtained from the lifetime study of FRWNFHRNTFFP-Toyopearl resin.

FIGS. 13A-13B: Druggability study of human Fab. Structure of human (A) Fab-κ (PDB ID: 4NYL, 5176, 5VZX, 5VL3, 5DK3, 5J13, 4QXG, 4G5Z, 5WUV, 3NFS, 3NFP, 5X8M, 4G6K, 3GIZ, 20SL, 5DK3, 5J13, 4QXG, 4G5Z, 5WUV, 3NFS, 3NFP, 5X8M, 4G6K, 3GIZ, and 2OSL) and (B) Fab-λ (PDB ID: 5L6Y, 4OD2, 5NGV, 5N2K); the full IgG scaffold is in white cartoon, the V_{H} and C_{H}1 domains are in teal cartoon, and the V_{L} and C_{L} domains are in pink cartoon. The insets present the corresponding putative binding site 1 (green mesh), site 2 (yellow-orange mesh), site 3 (wheat mesh), site 4 (blue mesh), site 5 (sand mesh), site 6 (pink mesh), site 7 (yellow mesh), and site 8 (orange mesh) identified on the homology structures of Fab-κ and Fab-λ using SiteMap.

FIGS. 14A-14B: Chromatograms obtained by loading (1) human polyclonal Fab at 1 mg/mL in CHO-S cell culture fluid with a HCP titer of 0.135 mg/mL, (2) Certolizumab Fab at 1 mg/mL in a null CHO-S cell culture fluid with a HCP titer of 0.109 mg/mL, or (3) an engineered monoclonal Fab-κ at 0.171 mg/mL in a clarified CHO-K1 cell culture harvest with a HCP titer of 0.157 mg/mL on (A) FRWNFHRNTFFP-Toyopearl resin and (B) Protein L-agarose resin.

FIG. 15: Chromatograms obtained by loading 0.1 mL of 1 mg/mL solutions of human polyclonal Fab from different manufacturers (A Athens research and technology, B Rockland Laboratories, C Fab lambda from Bethyl Laboratories, D Fab kappa from Bethyl Laboratories) onto FRWNFHRNTFFP-Toyopearl resin at the residence time of 2 min. Elution of bound Fab was conducted using 0.2 M acetate buffer at pH 4. Samples were non-diafiltered which means storage excipients with a UV 280 signal will show up in the flow-through.

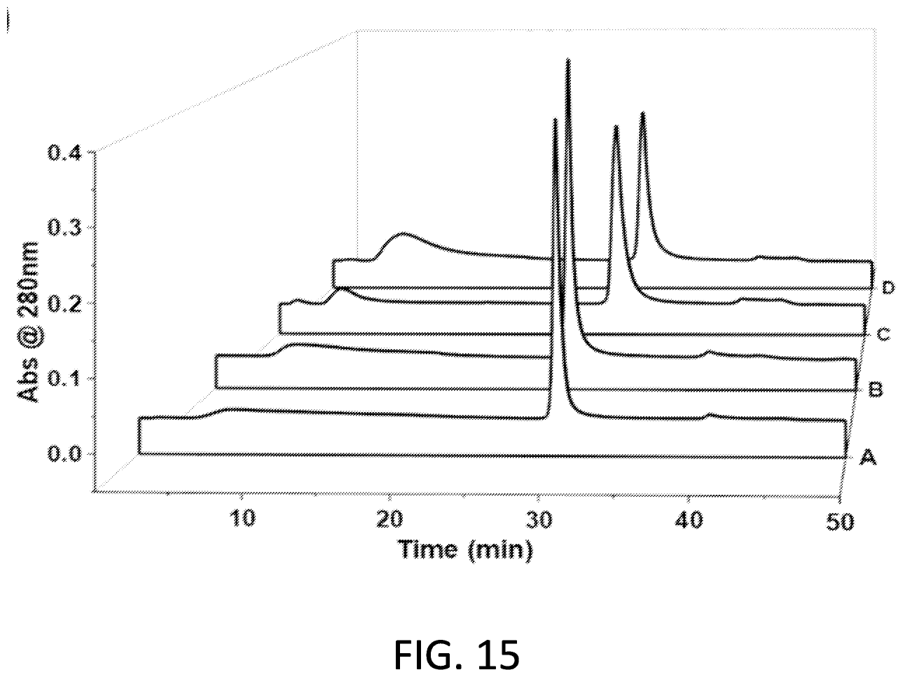

FIGS. 16A-16C: SEC chromatograms of (A) the feedstock (black) containing IgG in a P. pastoris perfusate and the flow-through (red) and elution (blue) fractions obtained by injecting 4.8 mL of feedstock in a 0.5 mL column packed with FRWNFHRNTFFP-Toyopearl resin; (B) the model feedstock (black) comprising a P. pastoris perfusate containing human IgG at 0.5 mg/mL, the flow-through (red) and elution (blue) fractions obtained by injecting 4.8 mL of feedstock in a 0.5 mL column packed with MabSelect Sure LX resin; (C) the feedstock (black) containing IgG in a P. pastoris perfusate, the flow-through (red) and elution (blue) fractions obtained by injecting 4.8 mL of feedstock in a 0.5 mL column packed with LigaTrap Human IgG resin.

FIGS. 17A-17C: Chromatograms obtained by loading a P. pastoris perfusate containing human IgG at 0.5 mg/mL (load volume: 4.8 mL; column volume: 0.5 mL) using (A) FRWNFHRNTFFP-Toyopearl, (B) MabSelect SuRe LX, and (C) LigaTrap Human IgG resin.

FIG. 18: Breakthrough curves of human IgG obtained by loading a P. pastoris perfusate (IgG titer: 0.5 mg/mL) on FRWNFHRNTFFP-Toyopearl resin at a residence time (RT) of 2 min.

FIG. 19: Chromatogram obtained by loading 20 mL of P. pastoris perfusate containing scFv at 0.028 mg/mL onto a 0.5 mL column packed with FRWNFHRNTFFP-Toyopearl resin. Collected fractions are marked that correspond to lanes analyzed in FIG. 20.

FIG. 20: Non-reducing SDS-PAGE analysis of the chromatographic fractions collected during the chromatographic purification of scFv from P. pastoris perfusate using FRWNFHRNTFFP-Toyopearl resin. Lane 1: molecular weight ladder; Lane 2: P. pastoris perfusate containing scFv; Lane 3: flow-through fraction part 1; Lane 4: flow-through fraction part 2; Lane 5: Wash fraction part 1; Lane 6: Wash fraction part 2; Lane 7: Elution.

FIG. 21: chromatogram obtained by loading 20 mL of P. pastoris perfusate containing scFv at 0.028 mg/mL onto a 0.5 mL column packed with FRWNFHRNTFFP-Toyopearl. Collected fractions are marked that correspond to lanes analyzed in FIG. 22.

FIGS. 22A-22B: Non-reducing SDS-PAGE analysis of (A) non-concentrated fractions and (B) fractions concentrated 20×, from scFv purification by FRWNFHRNTFFP-Toyopearl. Lanes identify for both (A) and (B) is as follows. Lane 1: molecular weight ladder; Lane 2: P. pastoris perfusate containing scFv; Lane 3: flow-through fraction part 1; Lane 4: flow-through fraction part 2; Lane 5: flow-through fraction part 3; Lane 6: wash fraction; Lane 7: elution part 1; Lane 8: elution part 2; Lane 9: elution part 3.

### DETAILED DESCRIPTION

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “peptide” and “polypeptide,” unless otherwise specified, generally refer to polymer compounds of two or more amino acids joined through the main chain by peptide amide bonds (—C(O)NH—). The term “peptide” typically refers to short amino acid polymers (e.g., chains having fewer than 25 amino acids), whereas the term “polypeptide” typically refers to longer amino acid polymers (e.g., chains having more than 25 amino acids).

As used herein, “sequence identity” generally refers to the degree two polymer sequences (e.g., peptide, polypeptide, nucleic acid, etc.) have the same sequential composition of monomer subunits. The term “sequence similarity” refers to the degree with which two polymer sequences (e.g., peptide, polypeptide, nucleic acid, etc.) have similar polymer sequences. For example, similar amino acids are those that share the same biophysical characteristics and can be grouped into the families, e.g., acidic amino acids (e.g., aspartate, glutamate), basic amino acids (e.g., lysine, arginine, histidine), non-polar amino acids (e.g., alanine, valine, leucine, isoleucine, proline, phenylalanine, methionine, tryptophan) uncharged polar amino acids amino acids amino acids (e.g., glycine, asparagine, glutamine, cysteine, serine, threonine, tyrosine), and aromatic amino acids (tryptophan, phenylalanine, tyrosine, histidine).

The “percent sequence identity” (or “percent sequence similarity”) is calculated by: (1) comparing two optimally aligned sequences over a window of comparison (e.g., the length of the longer sequence, the length of the shorter sequence, a specified window), (2) determining the number of positions containing identical (or similar) monomers (e.g., same amino acids occurs in both sequences, similar amino acid occurs in both sequences) to yield the number of matched positions, (3) dividing the number of matched positions by the total number of positions in the comparison window (e.g., the length of the longer sequence, the length of the shorter sequence, a specified window), and (4) multiplying the result by 100 to yield the percent sequence identity or percent sequence similarity. For example, if peptides A and B are both 20 amino acids in length and have identical amino acids at all but 1 position, then peptide A and peptide B have 95% sequence identity. If the amino acids at the non-identical position shared the same biophysical characteristics (e.g., both were acidic), then peptide A and peptide B would have 100% sequence similarity. As another example, if peptide C is 20 amino acids in length and peptide D is 15 amino acids in length, and 14 out of 15 amino acids in peptide D are identical to those of a portion of peptide C, then peptides C and D have 70% sequence identity, but peptide D has 93.3% sequence identity to an optimal comparison window of peptide C. For the purpose of calculating “percent sequence identity” (or “percent sequence similarity”) herein, any gaps in aligned sequences are treated as mismatches at that position.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “host cell protein” or “HCP” refers to any protein produced or encoded by the organism used to produce a recombinant polypeptide product and unrelated to the intended recombinant product. HCPs are generally undesirable in the final drug substance.

As used herein, a “mixture” comprises a target biologic of interest (for which purification is desired) and one or more contaminant or impurity. In some embodiments, the mixture is produced from a host cell or organism that expresses the protein of interest (either naturally or recombinantly). Such mixtures include, for example, cell cultures, cell lysates, and clarified bulk (e.g., clarified cell culture supernatant).

### 2. Compositions and Methods

Engineered multi-specific monoclonal antibodies (msAbs) and antibody domains and/or fragments offer valuable therapeutic options against metabolic disorders, aggressive cancers, and viral infections. The advancement in molecular design and recombinant expression of these next-generation drugs, however, is not equaled by the progress in downstream bioprocess technology. The purification of msAbs and their domains and/or fragments requires affinity adsorbents with orthogonal biorecognition of different portions of the antibody structure, namely its Fc and Fab domains or the C_{H}1-3 and C_{L} chains. Current adsorbents rely on protein ligands that, while featuring high binding capacity and selectivity, need harsh elution conditions and suffer from high cost, limited biochemical stability, and potential release of immunogenic fragments.

Responding to these challenges, experiments were conducted to identify novel peptide ligands that target different regions of human Fab and enable product release under mild conditions. The ligands were discovered by screening a focused library of 12-mer peptides against a feedstock comprising human Fab-κ and Chinese hamster ovary host cell proteins (CHO HCPs). The identified ligands were evaluated via equilibrium binding studies as well as molecular docking simulations, returning excellent values of binding capacity (Qₘₐₓ˜20 mg of Fab per mL of resin) and dissociation constant (K_{D}=2.16·10⁻⁶ M). One selected ligand, FRWNFHRNTFFP, and Protein L were further characterized by measuring the dynamic binding capacity (DBC_{10%}) at different residence times (RT) and performing the purification of engineered Fabs from CHO-K1 cell culture fluids. The peptide ligand featured DBC_{10%}˜6-16 mg/mL (RT of 2 min) and afforded values of yield (93-96%) and purity (89-96%) comparable to those provided by Protein L resins.

The current landscape of protein-based therapeutics features burgeoning groups of novel multi-specific mAbs whose purification requires multiple orthogonal steps of affinity chromatography. While Protein A-based resins can still be utilized in product capture, the combination of Fab domains of different subtypes within the multi-specific scaffold requires the use of Fab-targeting ligands capable of universal and subtype-specific biorecognition. Commercial products addressing these needs have been introduced over the past decade, but their adoption on the large scale is potentially limited by the high cost, harsh elution conditions, and limited safety and lifetime.

In response to these challenges, experiments were conducted to develop an ensemble of peptide ligands with broad and κ- or λ-selective affinity. As the first study investigating the activity of these ligands, embodiments of the present disclosure focused on fundamental characteristics such as the equilibrium and dynamic binding capacity of the peptide-based adsorbents, the binding affinity and selectivity, and the product yield and purity obtained by implementing these adsorbents in the purification of Fab from model and industrial cell culture fluids. With a DBC_{10%} comprised between 4.5 and 16.5 mg of Fab per mL of resin, the proposed peptide-based adsorbents perform comparably to commercial affinity resins for Fab purification. Similarly, the values of product yield and purity both global and HCP removal are also very high and seemingly unaffected by the complexity of the feedstock or the number of reuses.

While providing comparable performance to commercial counterparts, the proposed peptide-based resins would provide significant advantages in terms of operational costs. An analysis of production costs including raw materials, labor, and manufacturing under GMP conditions indicated, in fact, that adopting peptide-Toyopearl resins would be significantly financially advantageous: specifically, a FRWNFHRNTFFP-Toyopearl resin would cost ˜$6,500 per liter at the 10-liter scale and ˜$6,000 per liter of resin at the 100-liter scale.

In accordance with the above, embodiments of the present disclosure include novel peptide ligands capable of targeting the fragment antigen binding (Fab) domains and/or single-chain variable fragments (scFv) to facilitate the isolation and/or purification of Fab- and/or scFv-containing proteins or polypeptides (e.g., antibodies) from processing fluid streams. In some embodiments, the present disclosure provides a peptide ligand (or a plurality of peptide ligands) comprising no more than 12 amino acids and having the amino acid sequence X₁X₂X₃X₄X₅X₆X₇X₈X₉X₁₀X₁₁X₁₂, wherein: (i) positions X₁-X₅ comprise at least one positively charged amino acid or an aromatic amino acid; (ii) positions X₆-X₈ comprise at least one aromatic amino acid; and (iii) positions X₈-X₁₂ comprise at least one aliphatic amino acid or at least one aromatic amino acid. In some embodiments, the peptide ligand is capable of binding a target fragment antigen binding (Fab) domain of a Fab-containing protein or polypeptide. In some embodiments, the peptide ligand is capable of binding a single-chain variable fragment (scFv) of a scFv-containing protein or polypeptide.

As would be recognized by one of ordinary skill in the art based on the present disclosure, amino acid residues can be categorized based on certain biophysical characteristic. For example, similar amino acids can be grouped into the families, e.g., acidic amino acids (e.g., aspartate, glutamate), basic amino acids (e.g., lysine, arginine, histidine), non-polar amino acids (e.g., alanine, valine, leucine, isoleucine, proline, phenylalanine, methionine, tryptophan) uncharged polar amino acids amino acids amino acids (e.g., glycine, asparagine, glutamine, cysteine, serine, threonine, tyrosine), and aromatic amino acids (tryptophan, phenylalanine, tyrosine, histidine). In some embodiments, the peptide ligands of the present disclosure comprise at least one histidine residue or at least one arginine residue. In some embodiments, the peptide ligands of the present disclosure comprise at least one histidine residue and at least one arginine residue. In some embodiments, the peptide ligand(s) of the present disclosure comprises one or more of alanine (A), glutamic acid (E), phenylalanine (F), histidine (H), isoleucine (I), asparagine (N), proline (P), arginine (R), threonine (T), and tryptophan (W).

In some embodiments, the peptide ligand or plurality of peptide ligands of the present disclosure are capable of binding a target Fab comprising a lambda (λ) light chain isotype. In some embodiments, the peptide ligand(s) is capable of binding a target Fab comprising a kappa (κ) light chain isotype. In some embodiments, the peptide ligand(s) is capable of binding a target Fab comprising a lambda (λ) light chain isotype and a target Fab comprising a kappa (κ) light chain isotype. In some embodiments, the Fab comprises a single-chain variable fragment (scFv), and the peptide ligand is capable of binding the scFv. In some embodiments, the Fab and/or the scFv is comprised within a monoclonal antibody or a polyclonal antibody. In some embodiments, the Fab and/or the scFv is comprised within a mono-specific antibody or a multi-specific antibody. In some embodiments, the Fab and/or the scFv is comprised within a single-domain antibody, a humanized antibody, or a chimeric antibody. In some embodiments, the Fab and/or the scFv is comprised within a fusion protein.

In some embodiments, the peptide ligand(s) of the present disclosure exhibits a maximum equilibrium binding capacity (Qₘₐₓ) of at least about 15 mg of Fab/mL resin. In some embodiments, the peptide ligand(s) of the present disclosure exhibits a maximum equilibrium binding capacity (Qₘₐₓ) of at least about 20 mg of Fab/mL resin. In some embodiments, the peptide ligand(s) of the present disclosure exhibits a maximum equilibrium binding capacity (Qₘₐₓ) of at least about 25 mg of Fab/mL resin. In some embodiments, the peptide ligand(s) of the present disclosure exhibits a maximum equilibrium binding capacity (Qₘₐₓ) of at least about 30 mg of Fab/mL resin. In some embodiments, the peptide ligand(s) of the present disclosure exhibits a maximum equilibrium binding capacity (Qₘₐₓ) of at least about 35 mg of Fab/mL resin. In some embodiments, the peptide ligand(s) of the present disclosure exhibits a maximum equilibrium binding capacity (Qₘₐₓ) of at least about 40 mg of Fab/mL resin. In some embodiments, the peptide ligand(s) of the present disclosure exhibits a maximum equilibrium binding capacity (Qₘₐₓ) of at least about 45 mg of Fab/mL resin. In some embodiments, the peptide ligand(s) of the present disclosure exhibits a maximum equilibrium binding capacity (Qₘₐₓ) of at least about 50 mg of Fab/mL resin.

In some embodiments, the peptide ligand(s) of the present disclosure exhibits a disassociation constant (K_{D}) less than or equal to about 5×10⁻⁵ M. In some embodiments, the peptide ligand(s) exhibits a disassociation constant (K_{D}) less than or equal to about 5×10⁻⁶ M. In some embodiments, the peptide ligand(s) exhibits a disassociation constant (K_{D}) less than or equal to about 5×10⁻⁷ M. In some embodiments, the peptide ligand(s) exhibits a disassociation constant (K_{D}) less than or equal to about 5×10⁻⁸ M. In some embodiments, the peptide ligand(s) exhibits a disassociation constant (K_{D}) less than or equal to about 5×10⁻⁹ M. In some embodiments, the peptide ligand(s) exhibits a disassociation constant (K_{D}) less than or equal to about 5×10⁻¹⁰ M.

In some embodiments, the peptide ligand(s) of the present disclosure exhibits a dynamic binding capacity (DBC_{10%}) from about 5 mg/mL to about 20 mg/mL. In some embodiments, the peptide ligand(s) exhibits a dynamic binding capacity (DBC_{10%}) from about 10 mg/mL to about 20 mg/mL. In some embodiments, the peptide ligand(s) exhibits a dynamic binding capacity (DBC_{10%}) from about 15 mg/mL to about 20 mg/mL. In some embodiments, the peptide ligand(s) exhibits a dynamic binding capacity (DBC_{10%}) from about 5 mg/mL to about 15 mg/mL. In some embodiments, the peptide ligand(s) exhibits a dynamic binding capacity (DBC_{10%}) from about 5 mg/mL to about 10 mg/mL. In some embodiments, the peptide ligand(s) exhibits a dynamic binding capacity (DBC_{10%}) from about 10 mg/mL to about 15 mg/mL.

In some embodiments, the peptide ligand(s) of the present disclosure comprises an amino acid sequence having at least 70% sequence identity with any of SEQ ID NOs: 1-19. In some embodiments, the peptide ligand(s) of the present disclosure comprises an amino acid sequence having at least 75% sequence identity with any of SEQ ID NOs: 1-19. In some embodiments, the peptide ligand(s) of the present disclosure comprises an amino acid sequence having at least 80% sequence identity with any of SEQ ID NOs: 1-19. In some embodiments, the peptide ligand(s) of the present disclosure comprises an amino acid sequence having at least 85% sequence identity with any of SEQ ID NOs: 1-19. In some embodiments, the peptide ligand(s) of the present disclosure comprises an amino acid sequence having at least 90% sequence identity with any of SEQ ID NOs: 1-19. In some embodiments, the peptide ligand(s) of the present disclosure comprises an amino acid sequence having at least 91% sequence identity with any of SEQ ID NOs: 1-19. In some embodiments, the peptide ligand(s) of the present disclosure comprises an amino acid sequence having at least 92% sequence identity with any of SEQ ID NOS: 1-19. In some embodiments, the peptide ligand(s) of the present disclosure comprises an amino acid sequence having at least 93% sequence identity with any of SEQ ID NOs: 1-19. In some embodiments, the peptide ligand(s) of the present disclosure comprises an amino acid sequence having at least 94% sequence identity with any of SEQ ID NOs: 1-19. In some embodiments, the peptide ligand(s) of the present disclosure comprises an amino acid sequence having at least 95% sequence identity with any of SEQ ID NOs: 1-19. In some embodiments, the peptide ligand(s) of the present disclosure comprises an amino acid sequence having at least 96% sequence identity with any of SEQ ID NOs: 1-19. In some embodiments, the peptide ligand(s) of the present disclosure comprises an amino acid sequence having at least 97% sequence identity with any of SEQ ID NOs: 1-19. In some embodiments, the peptide ligand(s) of the present disclosure comprises an amino acid sequence having at least 98% sequence identity with any of SEQ ID NOs: 1-19. In some embodiments, the peptide ligand(s) of the present disclosure comprises an amino acid sequence having at least 99% sequence identity with any of SEQ ID NOs: 1-19. In some embodiments, the peptide ligand(s) of the present disclosure comprises an amino acid sequence having 100% sequence identity with any of SEQ ID NOs: 1-19.

In some embodiments, the peptide ligand(s) of the present disclosure comprises an amino acid sequence having at least 70% sequence identity with SEQ ID NO: 3. In some embodiments, the peptide ligand(s) of the present disclosure comprises an amino acid sequence having at least 75% sequence identity with SEQ ID NO: 3. In some embodiments, the peptide ligand(s) of the present disclosure comprises an amino acid sequence having at least 80% sequence identity with SEQ ID NO: 3. In some embodiments, the peptide ligand(s) of the present disclosure comprises an amino acid sequence having at least 85% sequence identity with SEQ ID NO: 3. In some embodiments, the peptide ligand(s) of the present disclosure comprises an amino acid sequence having at least 90% sequence identity with SEQ ID NO: 3. In some embodiments, the peptide ligand(s) of the present disclosure comprises an amino acid sequence having at least 91% sequence identity with SEQ ID NO: 3. In some embodiments, the peptide ligand(s) of the present disclosure comprises an amino acid sequence having at least 92% sequence identity with SEQ ID NO: 3. In some embodiments, the peptide ligand(s) of the present disclosure comprises an amino acid sequence having at least 93% sequence identity with SEQ ID NO: 3. In some embodiments, the peptide ligand(s) of the present disclosure comprises an amino acid sequence having at least 94% sequence identity with SEQ ID NO: 3. In some embodiments, the peptide ligand(s) of the present disclosure comprises an amino acid sequence having at least 95% sequence identity with SEQ ID NO: 3. In some embodiments, the peptide ligand(s) of the present disclosure comprises an amino acid sequence having at least 96% sequence identity with SEQ ID NO: 3. In some embodiments, the peptide ligand(s) of the present disclosure comprises an amino acid sequence having at least 97% sequence identity with SEQ ID NO: 3. In some embodiments, the peptide ligand(s) of the present disclosure comprises an amino acid sequence having at least 98% sequence identity with SEQ ID NO: 3. In some embodiments, the peptide ligand(s) of the present disclosure comprises an amino acid sequence having at least 99% sequence identity with SEQ ID NO: 3. In some embodiments, the peptide ligand(s) of the present disclosure comprises an amino acid sequence having 100% sequence identity with SEQ ID NO: 3.

In some embodiments, the peptide ligand(s) of the present disclosure comprises an amino acid sequence having at least 70% sequence identity with SEQ ID NO: 7. In some embodiments, the peptide ligand(s) of the present disclosure comprises an amino acid sequence having at least 75% sequence identity with SEQ ID NO: 7. In some embodiments, the peptide ligand(s) of the present disclosure comprises an amino acid sequence having at least 80% sequence identity with SEQ ID NO: 7. In some embodiments, the peptide ligand(s) of the present disclosure comprises an amino acid sequence having at least 85% sequence identity with SEQ ID NO: 7. In some embodiments, the peptide ligand(s) of the present disclosure comprises an amino acid sequence having at least 90% sequence identity with SEQ ID NO: 7. In some embodiments, the peptide ligand(s) of the present disclosure comprises an amino acid sequence having at least 91% sequence identity with SEQ ID NO: 7. In some embodiments, the peptide ligand(s) of the present disclosure comprises an amino acid sequence having at least 92% sequence identity with SEQ ID NO: 7. In some embodiments, the peptide ligand(s) of the present disclosure comprises an amino acid sequence having at least 93% sequence identity with SEQ ID NO: 7. In some embodiments, the peptide ligand(s) of the present disclosure comprises an amino acid sequence having at least 94% sequence identity with SEQ ID NO: 7. In some embodiments, the peptide ligand(s) of the present disclosure comprises an amino acid sequence having at least 95% sequence identity with SEQ ID NO: 7. In some embodiments, the peptide ligand(s) of the present disclosure comprises an amino acid sequence having at least 96% sequence identity with SEQ ID NO: 7. In some embodiments, the peptide ligand(s) of the present disclosure comprises an amino acid sequence having at least 97% sequence identity with SEQ ID NO: 7. In some embodiments, the peptide ligand(s) of the present disclosure comprises an amino acid sequence having at least 98% sequence identity with SEQ ID NO: 7. In some embodiments, the peptide ligand(s) of the present disclosure comprises an amino acid sequence having at least 99% sequence identity with SEQ ID NO: 7. In some embodiments, the peptide ligand(s) of the present disclosure comprises an amino acid sequence having 100% sequence identity with SEQ ID NO: 7.

In some embodiments, the peptide ligand(s) of the present disclosure comprises an amino acid sequence having at least 70% sequence identity with SEQ ID NO: 8. In some embodiments, the peptide ligand(s) of the present disclosure comprises an amino acid sequence having at least 75% sequence identity with SEQ ID NO: 8. In some embodiments, the peptide ligand(s) of the present disclosure comprises an amino acid sequence having at least 80% sequence identity with SEQ ID NO: 8. In some embodiments, the peptide ligand(s) of the present disclosure comprises an amino acid sequence having at least 85% sequence identity with SEQ ID NO: 8. In some embodiments, the peptide ligand(s) of the present disclosure comprises an amino acid sequence having at least 90% sequence identity with SEQ ID NO: 8. In some embodiments, the peptide ligand(s) of the present disclosure comprises an amino acid sequence having at least 91% sequence identity with SEQ ID NO: 8. In some embodiments, the peptide ligand(s) of the present disclosure comprises an amino acid sequence having at least 92% sequence identity with SEQ ID NO: 8. In some embodiments, the peptide ligand(s) of the present disclosure comprises an amino acid sequence having at least 93% sequence identity with SEQ ID NO: 8. In some embodiments, the peptide ligand(s) of the present disclosure comprises an amino acid sequence having at least 94% sequence identity with SEQ ID NO: 8. In some embodiments, the peptide ligand(s) of the present disclosure comprises an amino acid sequence having at least 95% sequence identity with SEQ ID NO: 8. In some embodiments, the peptide ligand(s) of the present disclosure comprises an amino acid sequence having at least 96% sequence identity with SEQ ID NO: 8. In some embodiments, the peptide ligand(s) of the present disclosure comprises an amino acid sequence having at least 97% sequence identity with SEQ ID NO: 8. In some embodiments, the peptide ligand(s) of the present disclosure comprises an amino acid sequence having at least 98% sequence identity with SEQ ID NO: 8. In some embodiments, the peptide ligand(s) of the present disclosure comprises an amino acid sequence having at least 99% sequence identity with SEQ ID NO: 8. In some embodiments, the peptide ligand(s) of the present disclosure comprises an amino acid sequence having 100% sequence identity with SEQ ID NO: 8.

In some embodiments, the peptide ligand(s) of the present disclosure comprises an amino acid sequence having at least 70% sequence identity with SEQ ID NO: 9. In some embodiments, the peptide ligand(s) of the present disclosure comprises an amino acid sequence having at least 75% sequence identity with SEQ ID NO: 9. In some embodiments, the peptide ligand(s) of the present disclosure comprises an amino acid sequence having at least 80% sequence identity with SEQ ID NO: 9. In some embodiments, the peptide ligand(s) of the present disclosure comprises an amino acid sequence having at least 85% sequence identity with SEQ ID NO: 9. In some embodiments, the peptide ligand(s) of the present disclosure comprises an amino acid sequence having at least 90% sequence identity with SEQ ID NO: 9. In some embodiments, the peptide ligand(s) of the present disclosure comprises an amino acid sequence having at least 91% sequence identity with SEQ ID NO: 9. In some embodiments, the peptide ligand(s) of the present disclosure comprises an amino acid sequence having at least 92% sequence identity with SEQ ID NO: 9. In some embodiments, the peptide ligand(s) of the present disclosure comprises an amino acid sequence having at least 93% sequence identity with SEQ ID NO: 9. In some embodiments, the peptide ligand(s) of the present disclosure comprises an amino acid sequence having at least 94% sequence identity with SEQ ID NO: 9. In some embodiments, the peptide ligand(s) of the present disclosure comprises an amino acid sequence having at least 95% sequence identity with SEQ ID NO: 9. In some embodiments, the peptide ligand(s) of the present disclosure comprises an amino acid sequence having at least 96% sequence identity with SEQ ID NO: 9. In some embodiments, the peptide ligand(s) of the present disclosure comprises an amino acid sequence having at least 97% sequence identity with SEQ ID NO: 9. In some embodiments, the peptide ligand(s) of the present disclosure comprises an amino acid sequence having at least 98% sequence identity with SEQ ID NO: 9. In some embodiments, the peptide ligand(s) of the present disclosure comprises an amino acid sequence having at least 99% sequence identity with SEQ ID NO: 9. In some embodiments, the peptide ligand(s) of the present disclosure comprises an amino acid sequence having 100% sequence identity with SEQ ID NO: 9.

In some embodiments, the peptide ligand(s) of the present disclosure comprises an amino acid sequence having at least 70% sequence identity with SEQ ID NO: 13. In some embodiments, the peptide ligand(s) of the present disclosure comprises an amino acid sequence having at least 75% sequence identity with SEQ ID NO: 13. In some embodiments, the peptide ligand(s) of the present disclosure comprises an amino acid sequence having at least 80% sequence identity with SEQ ID NO: 13. In some embodiments, the peptide ligand(s) of the present disclosure comprises an amino acid sequence having at least 85% sequence identity with SEQ ID NO: 13. In some embodiments, the peptide ligand(s) of the present disclosure comprises an amino acid sequence having at least 90% sequence identity with SEQ ID NO: 13. In some embodiments, the peptide ligand(s) of the present disclosure comprises an amino acid sequence having at least 91% sequence identity with SEQ ID NO: 13. In some embodiments, the peptide ligand(s) of the present disclosure comprises an amino acid sequence having at least 92% sequence identity with SEQ ID NO: 13. In some embodiments, the peptide ligand(s) of the present disclosure comprises an amino acid sequence having at least 93% sequence identity with SEQ ID NO: 13. In some embodiments, the peptide ligand(s) of the present disclosure comprises an amino acid sequence having at least 94% sequence identity with SEQ ID NO: 13. In some embodiments, the peptide ligand(s) of the present disclosure comprises an amino acid sequence having at least 95% sequence identity with SEQ ID NO: 13. In some embodiments, the peptide ligand(s) of the present disclosure comprises an amino acid sequence having at least 96% sequence identity with SEQ ID NO: 13. In some embodiments, the peptide ligand(s) of the present disclosure comprises an amino acid sequence having at least 97% sequence identity with SEQ ID NO: 13. In some embodiments, the peptide ligand(s) of the present disclosure comprises an amino acid sequence having at least 98% sequence identity with SEQ ID NO: 13. In some embodiments, the peptide ligand(s) of the present disclosure comprises an amino acid sequence having at least 99% sequence identity with SEQ ID NO: 13. In some embodiments, the peptide ligand(s) of the present disclosure comprises an amino acid sequence having 100% sequence identity with SEQ ID NO: 13.

In some embodiments, the peptide ligand(s) of the present disclosure comprises a linker. In some embodiments, the linker is capable of binding to a solid support. In some embodiments, the linker is bound to the C-terminus of the peptide ligand, and wherein the linker comprises a Glyn or a [Gly-Ser-Gly]ₘ, wherein 6≥n≥1 and 3≥m≥1. In some embodiments, the peptide ligand is bound to a solid support. In some embodiments, the solid support comprises a non-porous or porous particle, a membrane, a polymer surface, a fiber or a woven or non-woven fibermat, a hydrogel, a microplate, and/or a microfluidic device. In some embodiments, the porous particle is a chromatograph resin. In some embodiments, the solid support comprises polyacrylate, polyacrylamide, poly-ether, polyolefin, polyester, polysaccharide, iron oxide, silica, titania, and/or zirconia.

In accordance with the above, embodiments of the present disclosure include a composition for purifying a target biologic (e.g., a Fab-containing protein or polypeptide and/or a scFv-containing protein or polypeptide) from a fluid (e.g., a processing fluid stream) using any of the peptide ligands described herein. In some embodiments, the fluid is a cell culture fluid. In some embodiments, the fluid comprises a supernatant and/or a cellular lysate. In some embodiments, the fluid is derived from CHO cells. In some embodiments, the CHO cells are selected from the group consisting of: CHO-DXB11 cells, CHO-K1 cells, CHO-DG44 cells, and CHO-S cells, or any derivatives or variants thereof. In some embodiments, the biological fluid is derived from HEK293 cells. In some embodiments, the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof. In some embodiments, the biological fluid is derived from yeast cells or fungal cells. In some embodiments, the yeast cells are Pichia pastoris cells.

In some embodiments, the fluid comprises a pH from about 3.0 to about 9.0. In some embodiments, the fluid comprises a pH from about 4.0 to about 9.0. In some embodiments, the fluid comprises a pH from about 5.0 to about 9.0. In some embodiments, the fluid comprises a pH from about 6.0 to about 9.0. In some embodiments, the fluid comprises a pH from about 7.0 to about 9.0. In some embodiments, the fluid comprises a pH from about 8.0 to about 9.0. In some embodiments, the fluid comprises a pH from about 3.0 to about 8.0. In some embodiments, the fluid comprises a pH from about 3.0 to about 7.0. In some embodiments, the fluid comprises a pH from about 3.0 to about 6.0. In some embodiments, the fluid comprises a pH from about 3.0 to about 5.0. In some embodiments, the fluid comprises a pH from about 3.0 to about 4.0. In some embodiments, the fluid comprises a pH from about 4.0 to about 8.0. In some embodiments, the fluid comprises a pH from about 5.0 to about 7.0.

In some embodiments, the target biologic is at least one of a monoclonal antibody or a polyclonal antibody; a mono-specific antibody or a multi-specific antibody; a single-domain antibody, a humanized antibody, or a chimeric antibody; or a Fab-fusion protein.

Embodiments of the present disclosure also include an adsorbent for purifying a target biologic (e.g., a Fab-containing protein or polypeptide and/or a scFv-containing protein or polypeptide) from a fluid using any of the peptide ligands described herein. In some embodiments, an “adsorbent” is used herein generically to refer to the solid phase used in chromatography for which the mobile phase components exhibit a selective affinity. Because such affinity can take a variety of forms other than adsorption (including size exclusion or complexation), the term refers to solid phases that adsorb the components of a mixture and to solid phases that do not technically adsorb components from the mobile phase, but which nevertheless behave as an adsorbent by slowing the migration velocity of one component relative to another in a chromatographic system.

Embodiments of the present disclosure also include a method of purifying a target biologic (e.g., a Fab-containing protein or polypeptide and/or a scFv-containing protein or polypeptide) from a fluid. In accordance with these embodiments, the method includes contacting the fluid with any of the peptide ligands described herein, or any of the adsorbents described herein, under conditions sufficient for the peptide ligands or adsorbents to bind the target biologic; and eluting the target biologic from the peptide ligands. In some embodiments, “purified” when referring to a component or fraction indicates that its relative concentration (weight of component or fraction divided by the weight of all components or fractions in the mixture) is increased by at least 20%. In one series of embodiments, the relative concentration is increased by at least 40%, 50%, 60%, 75%, 100%, 150%, or 200%. A component or fraction can also be said to be purified when the relative concentration of components from which it is purified (weight of component or fraction from which it is purified divided by the weight of all components or fractions in the mixture) is decreased by at least 20%, 40%, 50%, 60%, 75%, 85%, 95%, 98% or 100%. In still another series of embodiments, the component or fraction is purified to a relative concentration of at least 50%, 65%, 75%, 85%, 90%, 97%, 98%, or 99%. When a component or fraction in one embodiment is “separated” from other components or fractions, it will be understood that in other embodiments the component or fraction is “purified” at levels provided herein.

In some embodiments, the conditions sufficient for the peptide ligands to bind the target biologic comprise use of a buffer comprising a pH ranging from about pH 6.0 to about pH 9.0. In some embodiments, the conditions sufficient for the peptide ligands to bind the target biologic comprise use of a buffer comprising a pH ranging from about pH 7.0 to about pH 9.0. In some embodiments, the conditions sufficient for the peptide ligands to bind the target biologic comprise use of a buffer comprising a pH ranging from about pH 8.0 to about pH 9.0. In some embodiments, the conditions sufficient for the peptide ligands to bind the target biologic comprise use of a buffer comprising a pH ranging from about pH 6.0 to about pH 8.0. In some embodiments, the conditions sufficient for the peptide ligands to bind the target biologic comprise use of a buffer comprising a pH ranging from about pH 6.0 to about pH 7.0. In some embodiments, the conditions sufficient for the peptide ligands to bind the target biologic comprise use of a buffer comprising a pH ranging from about pH 7.0 to about pH 8.0. In some embodiments, the conditions sufficient for the peptide ligands to bind the target biologic comprise use of a buffer comprising 10-100 mM phosphate buffer with 0-1 M NaCl at pH 6.5-8.5. In some embodiments, the conditions sufficient for the peptide ligands to bind the target biologic comprise use of a buffer comprising 10-100 mM Tris buffer with 0-1 M NaCl at pH 6.5-8.5.

In some embodiments, eluting the target biologic from the peptide ligands comprises use of a buffer comprising a pH ranging from about pH 2.0 to about pH 5.0. In some embodiments, eluting the target biologic from the peptide ligands comprises use of a buffer comprising a pH ranging from about pH 2.5 to about pH 5.0. In some embodiments, eluting the target biologic from the peptide ligands comprises use of a buffer comprising a pH ranging from about pH 3.0 to about pH 5.0. In some embodiments, eluting the target biologic from the peptide ligands comprises use of a buffer comprising a pH ranging from about pH 3.5 to about pH 5.0. In some embodiments, eluting the target biologic from the peptide ligands comprises use of a buffer comprising a pH ranging from about pH 4.0 to about pH 5.0. In some embodiments, eluting the target biologic from the peptide ligands comprises use of a buffer comprising a pH ranging from about pH 4.5 to about pH 5.0. In some embodiments, eluting the target biologic from the peptide ligands comprises use of a buffer comprising a pH ranging from about pH 2.0 to about pH 4.5. In some embodiments, eluting the target biologic from the peptide ligands comprises use of a buffer comprising a pH ranging from about pH 2.0 to about pH 4.0. In some embodiments, eluting the target biologic from the peptide ligands comprises use of a buffer comprising a pH ranging from about pH 2.0 to about pH 3.5. In some embodiments, eluting the target biologic from the peptide ligands comprises use of a buffer comprising a pH ranging from about pH 2.0 to about pH 3.0. In some embodiments, eluting the target biologic from the peptide ligands comprises use of a buffer comprising a pH ranging from about pH 2.0 to about pH 2.5. In some embodiments, eluting the target biologic from the peptide ligands comprises use of a buffer comprising a pH ranging from about pH 3.0 to about pH 4.0. In some embodiments, eluting the target biologic from the peptide ligands comprises use of a buffer comprising a pH ranging from about pH 2.5 to about pH 4.5. In some embodiments, eluting the target biologic from the peptide ligands comprises use of a buffer comprising a pH ranging from about pH 3.5 to about pH 4.5. In some embodiments, eluting the target biologic from the peptide ligands comprises use of a buffer comprising 0.1 M glycine pH 2.5-3.6. In some embodiments, eluting the target biologic from the peptide ligands comprises use of a buffer comprising 0.2 M acetate buffer pH 3.6-5.

In some embodiments, the methods described above for purifying a target biologic (e.g., a Fab-containing protein or polypeptide and/or a scFv-containing protein or polypeptide) from a fluid results in a yield for the target biologic of at least 70%. In some embodiments, the method results in a yield for the target biologic of at least 75%. In some embodiments, the method results in a yield for the target biologic of at least 80%. In some embodiments, the method results in a yield for the target biologic of at least 85%. In some embodiments, the method results in a yield for the target biologic of at least 90%. In some embodiments, the method results in a yield for the target biologic of at least 95%.

In some embodiments, the methods described above for purifying a target biologic (e.g., a Fab-containing protein or polypeptide and/or a scFv-containing protein or polypeptide) from a fluid results in a purity for the target biologic of at least 70%. In some embodiments, the method results in a purity for the target biologic of at least 75%. In some embodiments, the method results in a purity for the target biologic of at least 80%. In some embodiments, the method results in a purity for the target biologic of at least 85%. In some embodiments, the method results in a purity for the target biologic of at least 90%. In some embodiments, the method results in a purity for the target biologic of at least 95%.

### 3. Materials and Methods

Alexa Fluor 594 NHS ester (NHS-AF594), Alexa Fluor 488 TFP ester (TFP-AF488), N,N′-dimethylformamide (DMF), dichloromethane (DCM), glycine, glacial acetic acid, sodium acetate trihydrate, hydrochloric acid, Pierce BCA and microBCA kits, Invitrogen SilverQuest silver stain kit, and Pierce dye removal columns were purchased from ThermoFisher Scientific (Waltham, MA). The 3 kDa MWCO Amicon Ultra centrifugal filters, triisopropylsilane-silane (TIPS), ethanedithiol (EDT), Tween 20, aqueous NaOH, phosphate-buffered saline (PBS) at pH 7.4, the Kaiser test kit, and Aminomethyl-ChemMatrix resin were from Millipore-Sigma (Burlington, MA). Piperidine, diisopropylethylamine (DIPEA), trifluoroacetic acid (TFA), 1-methyl-2-pyrrolidinone (NMP), 2-(7-aza-1 Hbenzotriazol-1-yl)-1,1,3,3-tetramethyluronium hexafluorophosphate (HATU), and all Fmoc-protected amino acids were purchased from ChemImpex, Inc. (Wood Dale, IL). The human Fab-κ and Fab-λ standards were purchased from Bethyl Laboratories (Montgomery, TX) while human polyclonal Fab standards were sourced from Rockland Immunochemicals (Limerick, PA) and Athens Research & Technology (Athens, GA). Toyopearl AF-Amino-650M resin was purchased from Tosoh Bioscience (King of Prussia, PA). Microbore PEEK columns 30 mm long×2.1 mm I.D. were purchased from VICI Precision Sampling (Baton Rouge, LA). The BioResolve SEC mAb Column, 200 Å, 2.5 μm, 7.8×300 mm was from Waters Corporation. A 5 mL Protein A column and 1 mL Protein L column was purchased from Cytiva (Marlborough, MA). Protein L-agarose resin was purchased from Genscript (Piscataway, NJ). Certolizumab was purchased from MyBioSource (San Diego, CA). Clarified CHO-S and CHO-K1 cell culture fluids (CHO-S CCCF and CHO-K1 CCCF) were donated by the Biomanufacturing Training and Education Center (BTEC) at NC State University. The CHO ELISA kit was purchased from Cygnus technologies (Southport, NC). The Mini-PROTEIN TGX Precast gels were purchased from Bio-Rad (Hercules, CA).

Synthesis of the peptide library on ChemMatrix beads and selected peptides on Toyopearl resin. A solid-phase 12-mer One-Bead-One-Peptide (OBOP) library was synthesized on ChemMatrix resin by implementing the “split-couple-recombine” method developed by Lam et al. via Fmoc/tBu peptide synthesis strategy. Specifically, a library of ˜1012 unique sequences was synthesized on 500 mg of aminomethyl-ChemMatrix resin (functional density of 0.5-0.7 mmol per g resin) on a Syro I peptide synthesizer (Biotage, Uppsala, Sweden) using the protected amino acids Fmoc-Ala-OH, Fmoc-Glu(OtBu)-OH, Fmoc-Phe-OH, Fmoc-His(Trt)-OH, Fmoc-Ile-OH, Fmoc-Asn(Trt)-OH, Fmoc-Pro-OH, Fmoc-Arg(Pbf)-OH, Fmoc-Thr(tBu), and Fmoc-Trp (Boc)-OH. Each amino acid coupling reaction was performed for 15 min at 45° C. using a solution of 3 equivalents (3 eq.) of Fmoc-protected amino acid at 0.5 M in dry DMF, HATU (3 eq.) at 0.5 M in dry DMF, and DIPEA (6 eq.) at 0.5 M in dry NMP. The completion of each amino acid coupling was verified via Kaiser test, after which the Fmoc protecting group was removed by incubating the resin with 20% v/v piperidine in DMF for 20 min at room temperature. Upon completion of peptide elongation, the peptides were collectively deprotected via acidolysis using Reagent R (90% v/v TFA, 5% v/v thioanisole, 3% v/v EDT, and 2% v/v anisole) for 2.5 hours at room temperature under continuous end-over-end mixing. The deprotected library was finally washed with DCM and DMF, and stored in DMF at 4° C. The candidate peptides identified via library screening (Table 3) were synthesized on Toyopearl AF-Amino-650M resin using an Initiator+ Alstra (Biotage, Uppsala, Sweden) following the same procedure; these peptides were deprotected using a cocktail consisting of 95% v/v TFA, 2.5% v/v TIPS, and 2.5% v/v MilliQ water for 2.5 hours at room temperature and under constant mixing. The peptide-Toyopearl resins were washed with DCM and DMF, dried under N₂, and stored in 20% v/v aqueous ethanol at 4° C.

Fluorescent tagging of proteins. CHO cell culture fluid was concentrated using Amicon 3 kDa MWCO Ultra-15 centrifugal filter to a final HCP concentration of 2.1 mg/mL, as determined by Pierce BCA Protein Assay kit. Red-labeled Fab-κ was prepared by mixing 10 μL of a solution of NHS-AF594 at 10 mg/mL in anhydrous DMF with 100 μL of Fab-κ at 1 mg/mL in PBS at pH 7.4. Green-labeled CHO HCPs were prepared by mixing 10 μL of a solution of TFP-AF488 at 10 mg/mL in anhydrous DMF with 200 μL of diafiltered CHO HCPs at 2.1 mg/mL. Both samples were incubated for 1 hr at room temperature under end-over-end mixing. The unbound dyes were removed using Pierce dye removal columns. The final protein titers were quantified via BCA, while the absorbance of the solutions of AF594-Fab-κ and AF488-HCPs were measured by UV spectrophotometry at a wavelength of 590 nm and 490 nm, respectively, using a Synergy H1 plate reader (Biotek, Winooski, VT). From these values, the values of fluorophore-to-protein (F/P) ratio for the various labeled proteins were calculated using Equation 1: specifically, F/P ratios of 1.30 and 1.45 were obtained for AF594-Fab-κ and AF488-HCPs, respectively.

$$Labeling ratio = \frac{Abs of labeled protein}{ε \times protein titer ( M )} \times dilution factor \qquad Equation 1$$

Wherein the parameter & is the molar extinction coefficient of the dye.

Library screening. A screening mix was initially prepared comprising AF594-Fab-κ at 0.3 mg/mL and AF488-HCPs at 0.3 mg/mL in PBS at pH 7.4. Aliquots of 10 μL of OBOP library beads were equilibrated with PBS and incubated with 40 μL of screening mix for 2 hrs at 4° C. in the dark. Following copious rinsing with 0.5% v/v Tween 20 in PBS, aliquots of 3 μL of library beads were suspended in 50 mL of PBS. The beads were sorted using a microfluidic screening apparatus developed previously: specifically, (i) the beads were individually fed to a sorting chamber and imaged therein using a IX81 motorized trinocular inverted fluorescence phase contrast microscope (Olympus, Tokyo, Japan); (ii) the collected images were processed in real time using a customized Matlab code that quantifies the fluorescent emission of every bead and instruct the sorting chamber to discard the beads with insufficient emission as well as the green-only or red-and-green beads, and select red-only beads; the latter were (iii) withheld in the imaging chamber, where they were exposed to a flow of 0.2 M acetate buffer at pH 4 at 0.05 mL/min for 3 min, and subsequently re-imaged; (iv) the beads that maintained red fluorescence (no elution of bound Fab) were discarded, while the beads that lost >20-30% of the initial red fluorescence were selected. The positive beads were washed three times with 0.1 M glycine at pH 2.5 for 10 min at room temperature to remove all bound proteins, rinsed sequentially with MilliQ water and 20% v/v aqueous methanol, and finally analyzed via Edman degradation using a PPSQ-33a sequencer (Shimadzu, Kyoto, Japan) to sequence the candidate peptide ligands.

Dynamic binding of human Fab-κ and Fab-λ in non-competitive condition. The peptides listed in Table 3 were synthesized on Toyopearl resins as described herein, and the resulting adsorbents were packed into 0.1 mL (30×2.1 mm) columns, installed on a Waters Alliance 2695 HPLC system (Milford, MA), and equilibrated with PBS at pH 7.4. Binding of Fab was initially demonstrated by loading 50 μL of a solution of human polyclonal Fab at 2 mg/mL in PBS at the linear velocity of 87 cm/h. The subsequent chromatographic steps of washing with PBS, elution with 0.2 M acetate buffer pH 4, regeneration with 0.1 M glycine buffer pH 2.5, and equilibration with PBS were all performed at 348 cm/h. The dynamic binding protocol was repeated with 50 μL of human Fab-κ and Fab-λ, both at 1 mg/mL in PBS. The column effluent was continuously monitored at 280 nm by UV spectrophotometry. The yield was determined by peak area analysis and determined by taking the area of the elution peak divided by the flow-through, elution, and regeneration peak areas. Finally, the dynamic binding capacity at 10% flowthrough of FRWNFHRNTFFP-Toyopearl resin was measured via continuous loading of a solution of human polyclonal Fab at either 1, 2, or 5 mg/mL in PBS at the linear velocity of either 173, 87, or 35 cm/h, corresponding to the residence times (RT) of 1, 2, and 5 min, until reaching saturation of the adsorbent. The dynamic binding capacity of Protein L-agarose resin was measured via continuous loading of human polyclonal Fab at 1 mg/mL in PBS at the linear velocity of either 173 or 87 cm/h, corresponding to RT of 1 and 2 min, until reaching saturation of the adsorbent.

Equilibrium adsorption isotherms of human Fab on FRWNFHRNTFFP-Toyopearl, IIHRRAFWWFPN-Toyopearl, and Protein L-agarose resins. Individual aliquots of 6 μL of settled resin were incubated with 204 μL of Fab solutions at concentration ranging between 0.25 and 5 mg/mL in PBS for 3 hours at room temperature and under gentle mixing. The resin was pelleted by centrifugation and the supernatant was analyzed via UV spectrophotometry at 280 nm to measure the equilibrium concentration of unbound Fab (C*, mg of Fab per mL of solution). The corresponding values of resin-bound Fab (q, mg of Fab per mL of resin) were calculated via mass balance. The adsorption data (C*, q) were fitted against a Langmuir isotherm (Equation 2).

$$q = \frac{{Q}_{max} \times {C}^{*}}{{K}_{D} + {C}^{*}} \qquad Equation 2$$

Where the fitting parameters Qₘₐₓ and K_{D} are the maximum binding capacity of the resin (mg per mL of resin) and the Fab: peptide dissociation constant (affinity, μM).

In silico Fab: peptide binding studies. The crystal structures of human Fab-κ and Fab-λ listed in Table 1 were prepared using Protein Prep Wizard (PPW, Schrödinger, New York, NY) by correcting missing atoms and/or side chains (PRIME), removing salts and ligands, adding explicit hydrogens, and optimizing the hydrogen-bonding network; the minimization of the protein structure at the ionization state at pH 7.4 was performed using PROPKA. The curated structures were collated into homology models of Fab-κ and Fab-λ, which were analyzed using SiteMap to identify sites suitable for peptide binding, and the sites with high S-score (>0.8) and D-score (>0.9) were selected. Peptides IRHHNIWFNTIA-GSG, NNWWRHARIINA-GSG, RWTIWPPNWHPP-GSG, IIHRRAFWWFPN-GSG, and FRWNFHRNTFFP-GSG were constructed using the molecular editor Avogadro. The equilibration and production steps were performed using the Amber ff19SB force field. Every peptide was placed in a simulation box with periodic boundary and containing 1,000 water molecules (TIP3P model), and equilibrated with 10,000 steps of steepest gradient descent; the peptide was then heated to 300 K in an NVT ensemble for 250 ps with 1 fs time steps, and equilibrated to 1 atm with a 500-ps NPT simulation with 2 fs time steps. The production runs were performed in the NPT ensemble at T=300 K and P=1 atm using the Nose-Hoover thermostat and the Parrinello-Rahman barostat, respectively. The leap-frog algorithm was used to integrate the equations of motion, with integration steps of 2 fs, and the atomic coordinates were saved every 2 ps. All covalent bonds were constrained using the LINCS algorithm, the short-range electrostatic and Lennard-Jones interactions were calculated using cut-off values of 1.0 nm and 1.4 nm, and the particle-mesh Ewald method was utilized for long-range electrostatic interactions; the list of non-bonded interactions was updated every 5 fs using a cutoff of 1.4 nm. The peptides were then docked in silico against the binding sites identified on the homology models of Fab-κ and Fab-λ using the docking software HADDOCK (High Ambiguity Driven Protein-Protein Docking, v.2.4). The residues within the putative binding sites on Fab and the X₁-X₁₂ residues on the peptides were marked as “active”; the surrounding residues on Fab and the GSG segments on the C-terminus of the peptides were marked as “passive.” The docked Fab: peptide structures were grouped in clusters of up to 20 complexes based on Cα RMSD<5 Å and ranked using the dMM-PBSA score. Finally, the top Fab: peptide complexes were refined via 100-ns atomistic MD simulations to estimate the free energy of binding (ΔG_{B}).

Table 1. List of names, IgG class, Fab type, and corresponding PDB ID of therapeutic human Fab-κ and Fab-λ.

| Name | PDB ID | IgG class | Fab type | Name | PDB ID | IgG class | Fab type |
|---|---|---|---|---|---|---|---|
| Adalimumab | 4NYL | IgG₁ | κ-1 | Pembrolizumab | 5DK3 | IgG₄ | κ-3 |
| Cetuximab | 5I76 | IgG₁ | κ-1 | Tezepelumab | 5J13 | IgG₁ | κ-3 |
| Crenezumab | 5VZX | IgG₄ | κ-1 | Anifrolumab | 4QXG | IgG₁ | κ-4 |
| Epratuzumab | 5VL3 | IgG₁ | κ-1 | Canakinumab | 4G5Z | IgG₁ | κ-4 |
| Infliximab | 4G3Y | IgG₁ | κ-1 | Certolizumab | 5WUV | IgG₁ | κ-4 |
| Necitumumab | 6B3S | IgG₁ | κ-1 | Daclizumab | 3NFS | IgG₁ | κ-4 |
| Panitumumab | 5SX4 | IgG₂ | κ-1 | Daclizumab | 3NFP | IgG₁ | κ-4 |
| Sifalimumab | 4YPG | IgG₁ | κ-1 | Durvalumab | 5X8M | IgG₁ | κ-4 |
| Tanezumab | 4EDW | IgG₂ | κ-1 | Gevokizumab | 4G6K | IgG₂ | κ-4 |
| Trastuzumab | 4HKZ | IgG₁ | κ-1 | Ofatumumab | 3GIZ | IgG₄ | κ-4 |
| Atezolizumab | 5X8L | IgG₁ | κ-2 | Rituximab | 2OSL | IgG₁ | κ-4 |
| Basiliximab | 3IU3 | IgG₁ | κ-2 | Tralokinumab | 5L6Y | IgG₄ | λ-1 |
| Eculizumab | 5I5K | IgG₂ | κ-2 | Drozitumab | 4OD2 | IgG₁ | λ-2 |
| Nivolumab | 5GGQ | IgG₄ | κ-2 | Bimagrumab | 5NGV | IgG₁ | λ-5 |
| Onartuzumab | 4K3J | IgG₁ | κ-2 | Briakinumab | 5N2K | IgG₁ | λ-5 |
| Pertuzumab | 1S78 | IgG₁ | κ-2 | | | | |

Optimization of the binding buffer. Nine buffers were prepared using 50 mM phosphate as base buffer by adjusting the NaCl concentration to either 0 mM, 150 mM, or 500 mM, and the pH to either 6.5, 7.5, or 8.5. Nine feedstocks were prepared by diafiltering a clarified CHO-S cell culture fluid (CHO-S CCCF) comprising a therapeutic monoclonal Fab with a k light chain at 0.72 mg/mL and an HCP titer of 0.157 mg/mL against the corresponding binding buffers using 3 kDa centrifugal filters (Amicon, MilliporeSigma, Burlington, MA). FRWNFHRNTFFP-Toyopearl resin was wet packed into a 0.1 mL column and equilibrated in each binding buffer. A volume of 1 mL of diafiltered CCCF was then loaded on the column and processed following chromatographic purification protocol described herein. The effluent was continuously monitored at 280 nm by UV spectrophotometry, and the collected fractions were analyzed as described herein.

Purification of human Fab and a therapeutic Fab from CHO CCCFs. FRWNFHRNTFFP-Toyopearl resin was packed in a 0.1 mL column and equilibrated in 20 mM NaCl in 50 mM phosphate buffer at pH 7.5. Three feedstocks were prepared, namely (i) human Fab at 1 mg/mL in CHO-S CCCF at an HCP titer of 0.135 mg/mL, (ii) therapeutic Fab′ Certolizumab at 1 mg/mL CHO-S CCCF at an HCP titer of 0.109 mg/mL, and (iii) a CHO-K1 CCCF containing a monoclonal Fab-κ light chain at 0.171 mg/mL and featuring a HCP titer of 0.157 mg/mL. Diafiltered CCCF was loaded on the column and processed following chromatographic purification protocol described herein. To evaluate resin reusability, 20 cycles of purification of human polyclonal Fab from CHO-S cell culture supernatant were conducted using FRWNFHRNTFFP-Toyopearl resin. Following elution at pH 4 and cleaning at pH 2.5, resin regeneration was performed with strong denaturing conditions of 2 M urea in sodium acetate pH 4 buffer. The effluent was continuously monitored at 280 nm by UV spectrophotometry, and the collected fractions were analyzed as described herein.

Analytical characterization of chromatographic fractions. The purity of Fab in the collected fractions was evaluated via SDS-PAGE analysis under reducing conditions and non-reducing conditions, and size-exclusion chromatography (SEC). Mini-Protean TGX precast, 4-20% gels were utilized in SDS-PAGE and stained via Invitrogen SilverQuest Silver Staining Kit. The SEC HPLC analysis was conducted using a BioResolve SEC mAb Column, 200 Å, 2.5 μm, 7.8×300 mm Waters (Milford, MA) with 50 mM phosphate buffer at pH 7 as mobile phase. A sample volume of 20 μL was injected at the flowrate of 0.5 mL/min and the UV absorbance of the effluent was continuously monitored at 280 nm. The resulting SEC chromatograms were divided into the Fab peak area (MW˜50 kDa) and non-Fab peak area based on retention time. The purity (P, %) of Fab in the chromatographic fractions was calculated by SEC using Equation 3.

$$P ( \% ) = \frac{{A}_{Fab}}{{A}_{Fab} + {A}_{non - Fab}} \times 100 \% \qquad Equation 3$$

Where A_{Fab} and A_{non-Fab} are the values of the peak area for Fab and non-Fab proteins, respectively. The elution fractions were also analyzed using CHO-specific ELISA kits (Cygnus Technologies, Southport, SC) to measure the values of HCP titer and logarithmic removal value (HCP LRV).

Preparation of spiked feedstock. A wild-type X33 P. pastoris strain was obtained from ThermoFisher Scientific as an agar stab. This stab was used to create freezer stocks, which were stored at −80° C. A vial from the freezer stock was thawed, plated on a yeast extract peptone dextrose (YPD) agar plate, and incubated at 30° C. A single colony was then used to inoculate a 1 L vented shake flask containing 100 mL of Buffered Glycerol-complex Medium (BMGY), and the flask was incubated at 250 rpm at 30° C. A 300 mL bioreactor connected to a perfusion device was prepared and inoculated with cells harvested from the shake flask. After an initial growth phase, the perfusion system was activated, and fluid was continuously extracted from the bioreactor at a rate of 1.5 vessel volumes per day (VVD) while retaining the cells. Simultaneously to activating the perfusion system, a 2% v/v methanol feedstock containing IgG at 0.5 mg/mL began to be fed continuously into the bioreactor at 1.5 VVD. The perfusate, after neutralization to pH 7.4 and filtration through a 0.45 μm filter, was used as a feedstock for purification. A P. pastoris cell line obtained from the Biomanufacturing Training and Education center was used to produce scFv13R4 with Cymc and His tags under the same conditions.

Purification of IgG from P. pastoris perfusate. Three 0.5 mL columns (40×4 mm I.D.) were individually packed with FRWNFHRNTFFP-Toyopearl resin, MabSelect SuRe LX Protein A resin, and LigaTrap Human IgG resin, and equilibrated with PBS at pH 7.4. A volume of 4.8 mL of perfusate, featuring an IgG concentration of 0.5 mg/mL, was loaded onto the FRWNFHRNTFFP-Toyopearl resin, LigaTrap resin, and Protein A resin at a 2-minute residence time (RT). Subsequently, each resin was washed with PBS at pH 7.4. The bound IgG was eluted from the FRWNFHRNTFFP-Toyopearl resin and LigaTrap Human IgG resin using 0.2 M acetate buffer at pH 4; and from Protein A resin using 0.1 M citrate at pH 3. All resins were then regenerated with 0.1 M glycine buffer at pH 2.5. Washing, elution, and regeneration steps were all performed at 0.5 mL/min (231 cm/h), while the effluent was continuously monitored by UV spectrophotometry at 280 nm. Collected fractions were analyzed via size exclusion chromatography, analytical Protein G chromatography, and P. pastoris HCP ELISA. The yield was determined through analytical Protein G analysis, calculated by taking the feed and elution peak areas, multiplying them by the volume fed or collected, and then dividing the elution peak area result by the feed peak area result.

Dynamic binding capacity of IgG in competitive conditions. A 0.5 mL column (40×4 mm I.D.) was packed with FRWNFHRNTFFP-Toyopearl resin and equilibrated with PBS at pH 7.4. A volume of 35 mL of perfusate featuring an IgG concentration of 0.5 mg/mL was continuously loaded onto the column at a residence time of 2 minutes. The effluent was collected and apportioned in 750-μL fractions, which were analyzed via analytical Protein G chromatography to measure the IgG titer contained therein. The values of IgG titer in the effluent were utilized to calculate the dynamic IgG binding capacity at 10% breakthrough of FRWNFHRNTFFP-Toyopearl resin.

Purification of scFv from P. pastoris perfusate. A 0.5 mL column (40×4 mm I.D.) was packed with FRWNFHRNTFFP-Toyopearl resin and equilibrated with PBS at pH 7.4. A volume of 20 mL of perfusate, featuring a concentration of scFv of ˜28 μg/mL, was loaded onto the FRWNFHRNTFFP-Toyopearl resin at a 3-minute residence time (RT). Subsequently, the resin was washed with PBS at pH 7.4. The adsorbed scFv was eluted from the FRWNFHRNTFFP-Toyopearl resin with 0.2 M acetate buffer at pH 4. The resin was finally regenerated with 0.1 M glycine buffer at pH 2.5. Washing, elution, and regeneration steps were all performed at 0.5 mL/min (231 cm/h), while the effluent was continuously monitored by UV spectrophotometry at 280 nm. A second experiment was conducted where the same perfusate was clarified using dextran coated charcoal (DCC) at a 1% w/v solution (i.e., 1.5 grams DCC in 150 mL perfusate) for 5 minutes under continuous end-over-end mixing. The mixture was centrifuged at 1000 g for 5 minutes and the supernatant was filtered through a 0.22 μm membrane. A volume of 15 mL of clarified perfusate was processed using a 0.5 mL column packed with FRWNFHRNTFFP-Toyopearl resin: all chromatographic steps were conducted as described earlier in this section. The collected fractions were analyzed via non-reducing SDS-PAGE analysis after concentration and diafiltration to PBS at pH 7.4 by a 10 kDa MWCO spin concentrator.

### 4. Examples

The accompanying Examples are offered as illustrative as a partial scope and particular embodiments of the disclosure and are not meant to be limiting of the scope of the disclosure.

Embodiments of the present disclosure pertain to the de novo discovery of peptide ligands that target different regions of human Fab and enable product release under mild conditions. The ligands were discovered by selecting a designed ensemble of 12-mer peptides against a screening mix comprising human Fab-κ and Chinese hamster ovary host cell proteins (CHO HCPs). The identified ligands were evaluated in silico via molecular docking and dynamics simulations as well as in vitro via binding isotherm studies to measure their Fab-binding capacity and affinity. One exemplary peptide, FRWNFHRNTFFP, was utilized as an affinity ligand to purify engineered Fabs from CHO-K1 cell culture fluids.

### Example 1

Library design and screening. IgG-type antibodies feature a remarkable biomolecular diversity, which is often overlooked in the Protein A-based platform processes that dominate downstream bioprocessing of therapeutic mAbs. The Fc (fragment crystallizable) portion of IgG is divided into four subclasses (IgG₁, IgG₂, IgG₃, and IgG₄), which, while highly homologous, differ by several amino acids in the hinge region or upper C_{H}2 domain. The Fab (fragment antigen-binding) domain comprises 4 sub-domains, namely the constant domains of the heavy and light chains (C_{H}1 and C_{L}) and the variable domains of the heavy and light chains (V_{H} and V_{L}). Two classes of C_{L} domain exist, namely K and A, which are distinct in V_{L}-K subclasses (κ1-κ4) and six V_{L}-λ subclasses (21-26). The combinations of Fc and Fab isotypes engender a wide diversity of antibodies, each with unique physiological or therapeutic roles. Currently, therapeutic mAbs are mostly of the IgG₁ isotype, owing to the strong interaction between Fc₁ and the Fcγ receptor (FcγR) displayed on immune cells (e.g., B lymphocytes, natural killer cells, and macrophages) as compared to the other subclasses. Similarly, the Fab make-up of mAbs is therapeutically relevant: among the 137 reported therapeutic mAbs of human IgG₁ isotype, 124 feature k light chains and only 13 have λ chains. Most importantly, significant therapeutic activity of combinations of a MAb-κ and a MAb-λ has been demonstrated in vivo in both preclinical and clinical studies. Therefore, it stands to reason that a Fab-targeting ligand should be capable of binding the light chain of both κ and λ types.

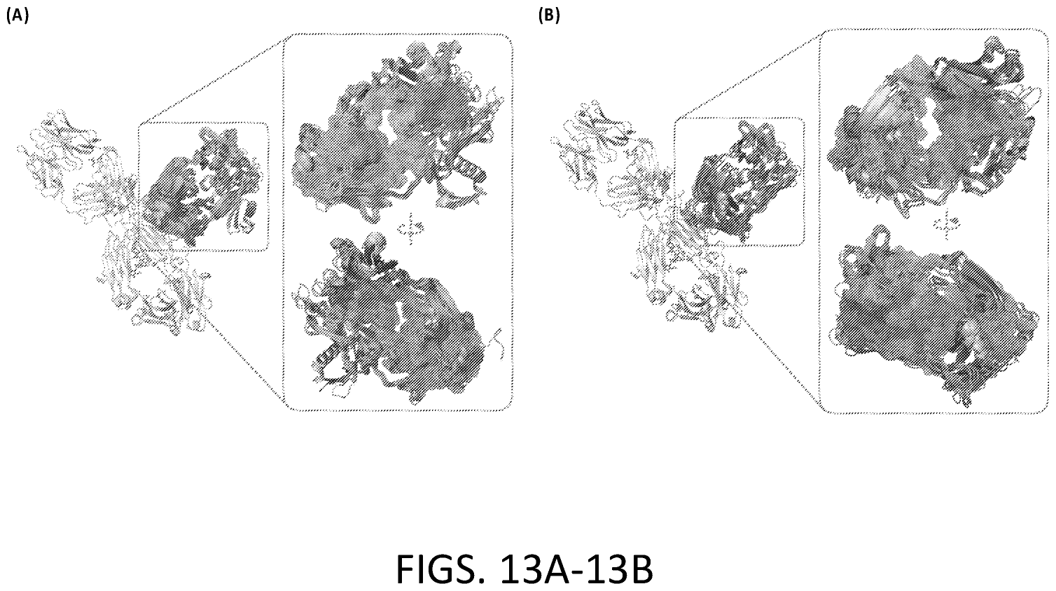

An in silico evaluation was performed of the homology structures of Fab-κ and Fab-λ derived by collating the crystal structures listed in Table 1. Using SiteMap, 7 putative pockets were identified on Fab-κ and 6 on Fab-λ, of which 3 are located on the C_{L} domain of Fab-κ and Fab-λ and 1 is located at the interface between the C_{H}1 and C_{L} domains; notably, these sites present morphological features and biophysical properties-including solvent-accessible area and volume, local isoelectric point, polarity, and grand average hydropathy-suitable to harbor peptides. The structure and properties of the selected target epitopes on the structures of Fab-κ and Fab-λ are collated in FIG. 13 and Table 2.

Table 2. Structural and biophysical properties of target sites on human Fab-κ and Fab-2. Properties of the putative binding sites identified by analyzing the homology structures of Fab-K (produced by collating PDB IDs 4NYL, 5176, 5VZX, 5VL3, 5DK3, 5J13, 4QXG, 4G5Z, 5WUV, 3NFS, 3NFP, 5X8M, 4G6K, 3GIZ, 20SL, 5DK3, 5J13, 4QXG, 4G5Z, 5WUV, 3NFS, 3NFP, 5X8M, 4G6K, 3GIZ, and 20SL) and Fab-λ (produced by collating PDB IDs 5L6Y, 4OD2, 5NGV, 5N2K) via SiteMap; the binding sites are labeled in FIG. 13.

| | Binding | PD | SASA | PV | | | |
|---|---|---|---|---|---|---|---|
| Fab | site | (Å) | (Å²) | (Å³) | pI | Polarity | GRAVY |
| Fab-κ | 1 | 13.40 | 168.08 | 1569.73 | 5.58 | 0.27 | −0.49 |
| | 2 | 13.16 | 153.10 | 1448.53 | 4.86 | 0.30 | −0.19 |
| | 3 | 12.51 | 154.74 | 1522.34 | 6.18 | 0.66 | −0.30 |
| | 4 | 12.69 | 159.71 | 1336.84 | 6.37 | 0.66 | −0.66 |
| | 5 | 13.78 | 164.99 | 1332.37 | 4.76 | 0.34 | −0.63 |
| | 6 | 13.98 | 162.56 | 1480.36 | 5.04 | 0.58 | −0.52 |
| | 7 | 12.99 | 174.97 | 1192.50 | 7.05 | 0.34 | −0.46 |
| | 8 | 13.23 | 160.03 | 1618.63 | 7.19 | 0.40 | −0.55 |
| Fab-λ | 1 | 12.72 | 153.94 | 1339.55 | 4.96 | 0.62 | −0.27 |
| | 2 | 13.55 | 172.34 | 1150.18 | 6.35 | 0.67 | −0.24 |
| | 3 | 13.53 | 156.26 | 1149.64 | 7.62 | 0.24 | −0.46 |
| | 4 | 14.45 | 147.88 | 1440.01 | 5.01 | 0.23 | −0.25 |
| | 5 | 13.59 | 156.74 | 1624.92 | 7.59 | 0.25 | −0.49 |
| | 6 | 13.71 | 171.49 | 1249.01 | 5.49 | 0.47 | −0.24 |
| | 7 | 13.47 | 145.44 | 1431.51 | 6.26 | 0.63 | −0.08 |
| | 8 | 13.40 | 168.08 | 1569.73 | 5.58 | 0.27 | −0.49 |

The analysis of the selected epitopes suggested the adoption of 12-mer linear peptides (X₁-X₁₂-GSG) as scaffolds to identify suitable Fab-binding ligands; and the use of alanine (A), glutamic acid (E), phenylalanine (F), histidine (H), isoleucine (I), asparagine (N), proline (P), arginine (R), threonine (T), and tryptophan (W) to populate the combinatorial positions Xᵢ; finally, the tripeptide spacer GSG (Gly-Ser-Gly) was introduced on the peptide C-terminus to promote the display of the variable segment X₁-X₁₂.

The peptide libraries were synthesized on ChemMatrix beads following the “split-couple-and-recombine” strategy introduced by Lam et al. ChemMatrix resin was adopted as library substrate owing to its high particle size, large porosity and pore diameter, low non-specific protein binding, and translucency, which make it an ideal substrate for selecting peptide ligands via fluorescence-based screening. The resultant one-bead-one-peptide (OBOP) library was selected against Fab-κ and Fab-λ using a microfluidic device for high-throughput screening of solid-phase peptide libraries. To select selective Fab-targeting peptides, the library was screened under competitive conditions using a screening mix formulated to mimic the feedstocks that access industrial purification pipelines: the human Fab, present at 0.3 mg/mL, is labeled with a red fluorophore; the host cell proteins (HCPs) produced by Chinese hamster ovary (CHO) cells, also at 0.3 mg/mL, are collectively labeled with a green fluorophore.

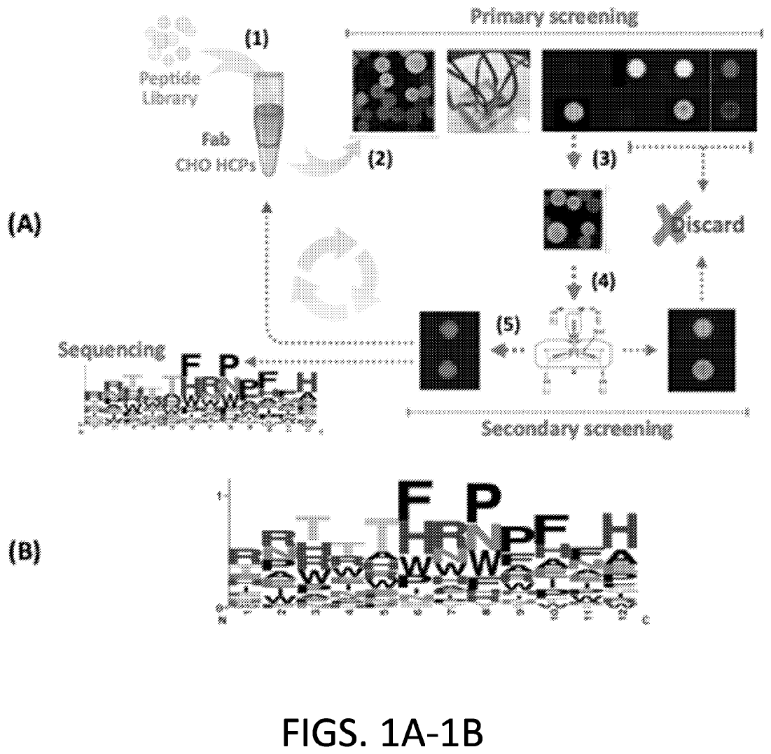

Following incubation with the screening mix, the peptide-ChemMatrix library beads are washed and fed to the screening device. This comprises a bead-imaging/sorting chamber placed in a fluorescence microscope and equipped with a camera for high-resolution imaging: the images of beads are analyzed to extract image metrics that correlate to the affinity of protein(s): ligand binding and automate the selection of positive beads (FIG. 1A). The beads that displayed insufficient red-fluorescence or a detectable green fluorescence-namely, those carrying peptides that target Fab with insufficient binding strength or selectivity-were discarded. Conversely, the beads with high intensity and radial distribution of red fluorescence were withheld in the imaging chamber and exposed to a flow of buffer adopted for Fab elution, namely 0.2 M sodium acetate at pH 4. Accordingly, every bead that displayed a measurable loss of red fluorescence (>20%) was selected as a positive lead. Including the step of target elution under desired conditions in library screening is a unique feature of the ligand selection method and is critical to ensure the selection of peptides that can function efficiently as affinity ligands in a purification pipeline of biological drugs.

This process returned 19 beads, which were analyzed via Edman degradation to identify the candidate Fab-binding peptides (Table 3).

Table 3. Fab-binding candidate peptides which were obtained by sequencing the positive beads from library screening with Fab-κ and CHO HCPs.

| SEQ ID | | | | | | | | | | | | |
|---|---|---|---|---|---|---|---|---|---|---|---|---|
| NO: | Sequence | | | | | | | | | | | |
| 1 | E | E | W | X | X | W | X | P | P | F | F | X |
| 2 | E | N | F | W | W | W | N | W | R | X | W | G |
| 3 | F | R | W | N | F | H | R | N | T | F | F | P |
| 4 | G | A | R | R | E | N | X | F | F | A | A | F |
| 5 | H | P | R | R | T | H | R | W | W | W | W | E |
| 6 | H | P | H | E | A | H | R | P | P | H | R | H |
| 7 | I | R | H | H | N | I | W | F | N | T | I | A |
| 8 | I | I | H | R | R | A | F | W | W | F | P | N |
| 9 | N | N | W | W | R | H | A | R | I | I | N | A |
| 10 | P | R | N | W | R | P | I | H | A | I | I | H |
| 11 | R | A | R | E | H | H | W | W | R | E | E | H |
| 12 | R | E | R | N | R | H | F | P | H | F | T | H |
| 13 | R | W | T | I | W | P | P | N | W | H | P | P |
| 14 | R | W | P | R | T | H | H | N | P | A | A | A |
| 15 | R | I | T | R | T | F | N | P | P | P | F | W |
| 16 | T | R | T | R | H | F | R | P | P | E | E | E |
| 17 | T | P | T | F | R | W | H | N | F | F | P | I |
| 18 | T | F | A | A | T | F | N | P | A | H | N | A |
| 19 | W | N | P | I | P | S | Y | Y | P | F | F | R |

The comparative sequence analysis reported in FIG. 1B indicates that the sequences are enriched in (i) polar, especially cationic, residues towards the N-terminus (X₁-X₅) and aliphatic and aromatic residues towards the C-terminus (X₈-X₁₂); and (ii) aromatic residues in the center (X₆-X₈); and (iii) histidine residues. The enrichment in His residues was believed to be a direct result of the conditions implemented during library screening. The imidazole pendant group of histidine, with a pKₐ of 6.0, is neutral at the pH 7.4 adopted for Fab binding, but becomes positively charged at the pH 4 adopted for Fab elution, and can promote dissociation of Fab via electrostatic repulsion; this has been verified during the in silico analysis of the Fab complexes formed by the lead peptide ligand FRWNFHRNTFFP. It therefore stands to reason that the presence of histidine residues in the identified sequences is strongly connected with the purposeful selection of candidate ligands that bind at pH 7.4 and elute at pH 4.

### Example 2

Secondary selection of Fab-binding peptide ligands via dynamic binding studies. The candidate ligands selected from library screening were conjugated to Toyopearl AF-Amino-650M resin and evaluated via Fab binding studies in dynamic mode. Toyopearl resin was selected for chromatographic evaluation owing to its low non-specific protein adsorption, low compressibility, and strong chemical stability. The residence time of 2 minutes (RT: 2 min) was adopted to mimic the loading conditions utilized in industrial bioprocessing. A solution of human polyclonal Fab at 2 mg/mL in PBS was adopted as feedstock to discern ligands that either bind all Fab or target selectively Fab-κ or Fab-λ; accordingly, the top ligands by elution peak area were subjected to further analysis with either pure k or λ subtypes. Finally, in line with library screening, A 0.2 M acetate buffer was utilized at pH 4 for Fab elution; this value of pH is sufficient to ensure high yield without compromising the integrity and bioactivity of the product. This represents a distinct advantage of peptide ligands with moderate affinity compared to high-affinity protein ligands; for comparison, Protein L as well as KappaSelect and LambdaFabSelect resins require elution at pH 2.5-3.

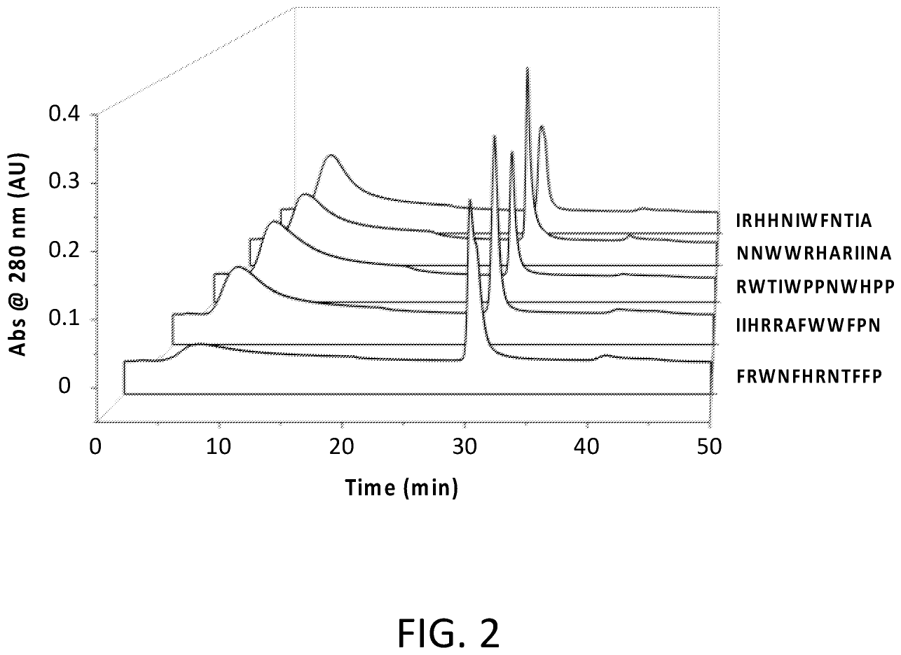

FIG. 2 reports the chromatograms obtained with IRHHNIWFNTIA-, NNWWRHARIINA-, RWTIWPPNWHPP-, IIHRRAFWWFPN-, and FRWNFHRNTFFP-Toyopearl resin. Notably, FRWNFHRNTFFP had a very low flow-through peak area demonstrating broad binding of Fab subtypes. Subsequent in silico studies indicated that the peptide targets a region in the C_{H}1 domain that is highly conserved among Fab-κ and Fab-2.

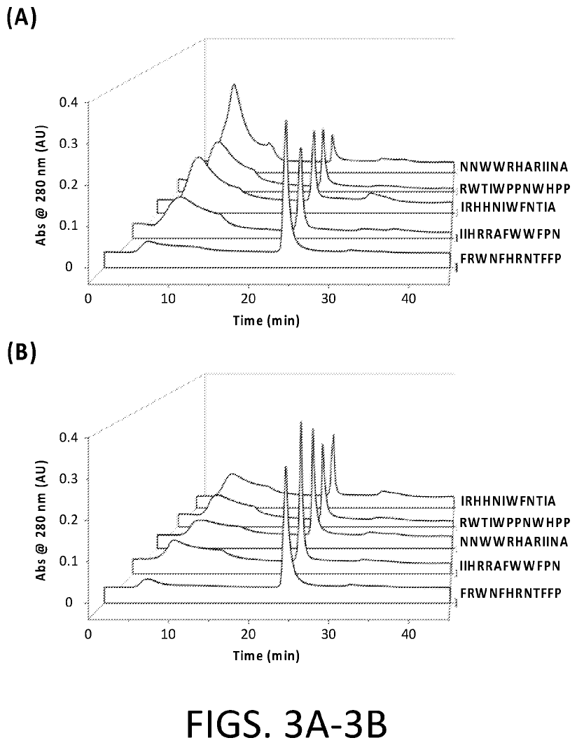

The chromatograms obtained by evaluating the binding of human Fab-κ or Fab-λ by the five peptide-Toyopearl resins and the resulting values of Fab yield are reported in FIG. 3 and Table 4, respectively.

Table 4. Yields of Fab-κ or Fab-λ obtained by loading Fab at 1 mg/mL in PBS on a 0.1 mL column packed with IRHHNIWFNTIA-, RWTIWPPNWHPP-, NNWWRHARIINA-, IIHRRAFWWFPN-, or FRWNFHRNTFFP-Toyopearl resin at the residence time of 2 min; Fab elution was performed at pH 4.

| Peptide | Fab-κ Yield | Fab-λ Yield |
|---|---|---|
| FRWNFHRNTFFP | 89% | 91% |
| IIHRRAFWWFPN | 55% | 66% |
| IRHHNIWFNTIA | 42% | 42% |
| RWTIWPPNWHPP | 40% | 53% |
| NNWWRHARIINA | 19% | 60% |

Among the candidate ligands, only FRWNFHRNTFFP and IIHRRAFWWFPN bound κ and λ Fabs comparably. The two sequences, however, differed appreciably in terms of product capture and recovery, with FRWNFHRNTFFP binding approximately 90% of the load and eluting completely the adsorbed Fab, whereas IIHRRAFWWFPN only bound approximately half of the loaded Fab. Notably, NNWWRHARIINA showed a distinct selectivity for Fab-λ, whose binding was found to be in 3:1 ratio with respect to Fab-κ; in future work, this sequence will serve as precursor to develop derivative ligands for the fractionation of human Fab or the purification of κ/λ) hybrid msAbs. The other two candidate ligands, IRHHNIWFNTIA and RWTIWPPNWHPP, afforded poor yield (<50%) of both κ and λ Fabs, and were therefore abandoned.

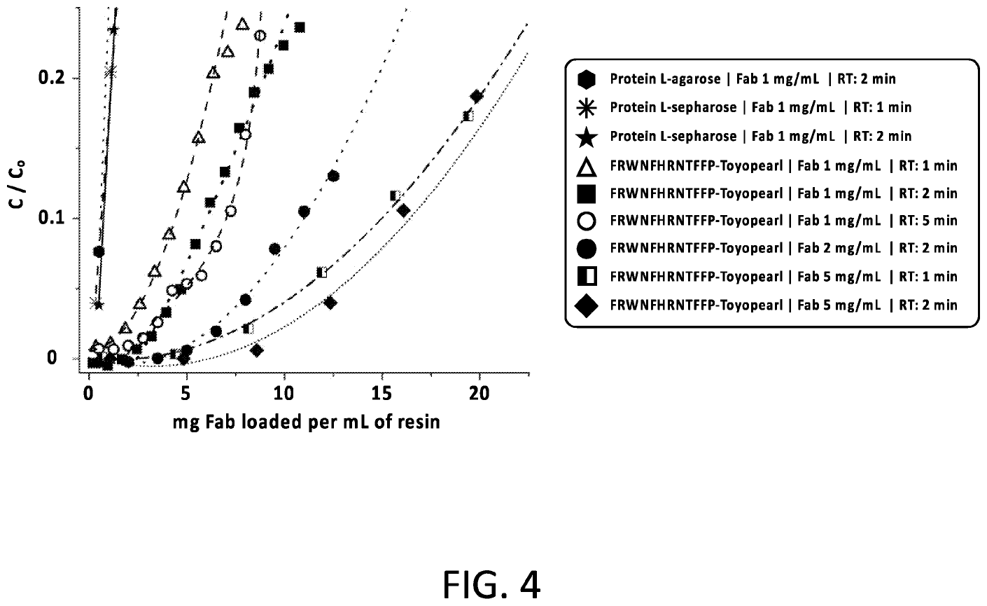

The dynamic binding capacity of FRWNFHRNTFFP-Toyopearl was then measured at 10% of breakthrough (DBC_{10%}) obtained by loading a solution of human polyclonal Fab at different concentrations in PBS—namely, 1, 2, and 5 mg/mL—at the RT of either 1, 2, or 5 min. The resulting breakthrough curves are reported in FIG. 4 and the corresponding values of DBC_{10%} are collated in Table 5.

Table 5. Values of dynamic Fab binding capacity (DBC_{10%}) of FRWNFHRNTFFP-Toyopearl and Protein L-based resins evaluated with Fab solutions in PBS at different titers and at different values of residence time (RT).

| Fab | | | | |
|---|---|---|---|---|
| Concen- | Residence | DBC_{10%} (mg Fab per mL of resin) | | |
| tration | time | FRWNFHRNTFFP- | Protein | Protein |
| (mg/mL) | (min) | Toyopearl | L-agarose | L-Sepharose |
| 1 mg/mL | 1 min | 4.5 | 0.6 | 0.5 |
| | 2 min | 6 | 0.5 | |
| | 5 min | 7 | | |
| 2 mg/mL | 2 min | 10.9 | | |
| 5 mg/mL | 1 min | 15.5 | | |
| | 2 min | 16.5 | | |

The low values of DBC_{10%} of Protein L-agarose resins, utilized here as controls, can be attributed to the flow through of Fab-κ2 and Fab-λ, which are not captured by Protein L. The values of dynamic capacity for FRWNFHRNTFFP varied rather widely with protein titer and residence time, ranging from 4.5 to 16.5 mg of Fab per mL of resin. The latter value, however, is in line with the binding capacity of the reference Protein L resins (i.e., 15 mg/mL static binding to 25 mg/mL dynamic binding at the RT of 4 minutes), and the reported values of KappaSelect (i.e., 15 mg/mL measured by amount of eluted protein from a KappaSelect column loaded to excess) and LambdaFabSelect (i.e., 20 mg/mL at the RT of 4 min) resins. Furthermore, the significantly lower cost of FRWNFHRNTFFP-Toyopearl resin compared to affinity adsorbents that employ protein ligands can offset the difference in binding capacity, thus making peptide-based adsorbents effective alternatives for downstream bioprocessing of Fab-based therapeutics.

The marked dependence of DBC_{10%} upon feed flowrate can be attributed to moderate adsorption kinetics (kₒₙ), which aligns with the moderate affinity (K_{D}=k_{off}/kₒₙ) of the peptide ligands. Integrating a Fab elution step under mild conditions (pH 4) in the library screening indeed biases the peptide selection towards ligands with moderate binding strength; this was also confirmed by the equilibrium binding studies presented herein. Furthermore, prior studies have linked ligand display—namely, density and orientation of the peptides on the resin's surface and inclusion of a spacer arm—to binding capacity;⁵⁸ this suggests that optimizing the design of peptide-based adsorbents can further increase DBC_{10%} to or above the values of commercial affinity adsorbents.

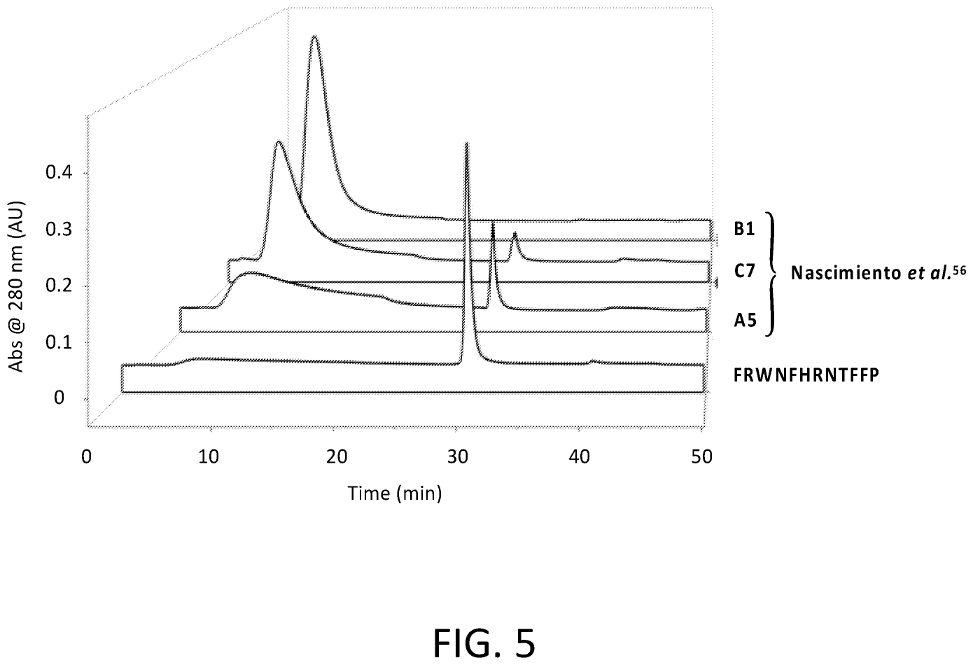

The lead ligand FRWNFHRNTFFP was then compared to three peptides identified by Nascimento et al. as ligands for monoclonal Fab-κ.⁵⁹ The team initially developed WIPNSEFEHERTK (B1) as the lead ligand to purify Fab-κ A from an E. coli lysate with 84% yield and purity above 90%. Peptide B1, however, demonstrated little-to-no affinity for two other monoclonal Fabs,⁵⁹ and, when evaluated in this study, it failed to bind human polyclonal Fab (FIG. 5). These results suggest that the binding site of B1 is located in the hypervariable region of Fab-K, such as the complementarity determining region (CDR) loops. With the purpose of identifying peptide ligands with broader Fab binding, Nascimento et al. developed variants HQNHHSTFRWEIY (C7) and WHYNWQDVSDRQ (A5). FIG. 5 shows that C7 and A5 indeed feature better Fab binding, although an appreciable amount of human Fab (79% and 61%, respectively), flowed through. For A5, the unbound fraction may be rich in Fab-λ, as the ligand variants were developed by selecting a peptide library against three monoclonal Fab-κ targets.

### Example 3

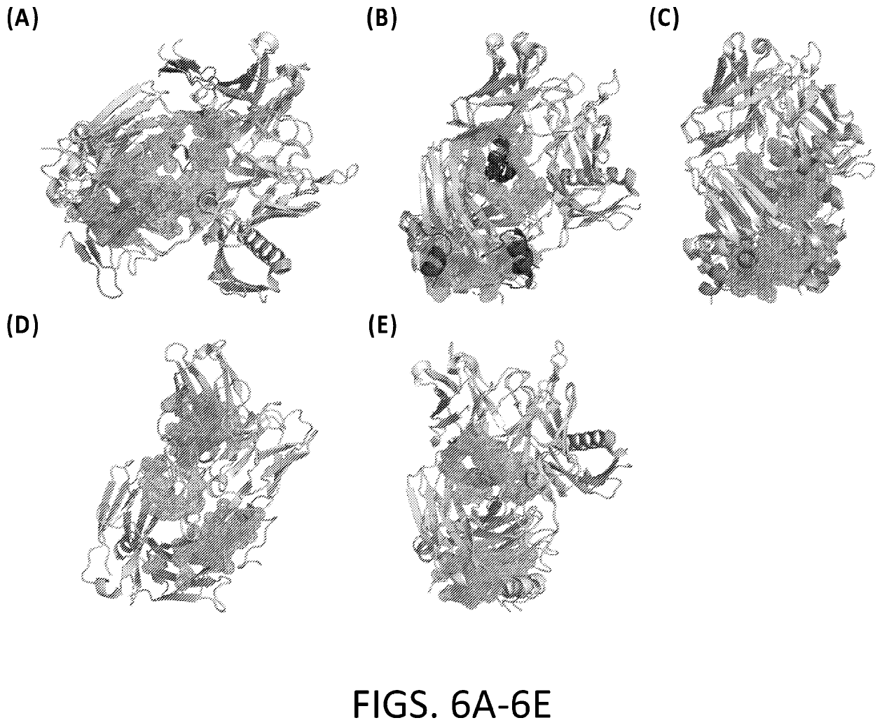

In silico and in vitro equilibrium binding studies of human Fab. The conditions implemented in library screening and the results of the dynamic binding studies indicate that the selected peptides target human Fab (i) with moderate affinity, thus enabling complete elution at pH 4, and (ii) at different segments of the light chain-heavy chain dimer, thus accomplishing either global or differential binding of Fab-κ and Fab-λ. To further investigate these phenomena, an in silico analysis was conducted of the binding site and energy of peptides IRHHNIWFNTIA, NNWWRHARIINA, RWTIWPPNWHPP, IIHRRAFWWFPN, and FRWNFHRNTFFP on the homology models of human Fab-κ and Fab-λ constructed by collating the crystal structures listed in Table 1. To this end, the secondary structures of the peptides were generated via MD simulations and docked on the putative binding sites identified for library design using HADDOCK v.2.4. To mimic the conjugation of the peptides on the surface of the chromatographic resin, the C-terminus of the above-listed sequences were derivatized with the tripeptide GSG and designated it as passive (i.e., not interacting with Fab). The resultant Fab: peptide complexes were refined via MD simulations in explicit solvent to calculate the binding free energy (ΔG_{b}) and ultimately the dissociation constant K_{D,in silico}. The resulting complexes formed by the selected peptides and Fab-κ and Fab-λ are reported in FIG. 6, while the corresponding values of K_{D,in silico} are collated in Table 6.

Table 6. Values of K_{D,in silico} calculated via molecular docking and dynamics of peptides FRWNFHRNTFFP-GSG, IIHRRAFWWFPN-GSG, IRHHNIWFNTIA-GSG, NNWWRHARIINA-GSG, and RWTIWPPNWHPP-GSG on the putative binding sites identified on the homology structures of Fab-κ and Fab-λ.

| | | FRWNFHRNTFFP- | IIHRRAFWWFPN- | IRHHNIWFNTIA- | NNWWRHARIINA- | RWTIWPPNWHPP- |
|---|---|---|---|---|---|---|
| Fab | Site | GSG | GSG | GSG | GSG | GSG |
| κ | 1 | 5.3-9.1 · 10⁻⁷ M | 4.1-7.7 · 10⁻⁶ M | 5.4-10.1 · 10⁻⁶ M | | 6.5-12.1 · 10⁻⁶ M |
| | 2 | | | [symbol] | | |
| | 3 | [symbol] | | 6.0-9.2 · 10⁻⁶ M | [symbol] | |
| | 4 | | | | | |
| | 5 | 1.2 · 10⁻⁶ M | 5.5-6.0 · 10⁻⁶ M | | [symbol] | 4.2-7.1 · 10⁻⁶ M |
| | 6 | | | | | [symbol] |
| | 7 | [symbol] | | | [symbol] | |
| | 8 | | | | | |
| λ | 1 | 5.1-9.1 · 10⁻⁷ M | 4.3-11.1 · 10⁻⁶ M | 5.5-10.1 · 10⁻⁶ M | | 6.5-8.9 · 10⁻⁶ M |
| | 2 | | | [symbol] | | |
| | 3 | [symbol] | | 6.0-8.2 · 10⁻⁶ M | 3.2 · 10⁻⁶ M | |
| | 4 | | | | | |
| | 5 | 1.05 · 10⁻⁶ M | 4.3-6.3 · 10⁻⁶ M | | [symbol] | 3.9-7.1 · 10⁻⁶ M |
| | 6 | | | | | [symbol] |
| | 7 | [symbol] | | | 6.4 · 10⁻⁶ M | |
| | 8 | | | | | |

As anticipated, these results indicate that the selected peptides bind Fab (i) with moderate affinity (K_{D,in silico}˜10⁻⁶ M; for reference the Fab: Protein L complex in PDB ID 4 HKZ features a K_{D,in silico} of 2.8 10⁻⁸ M); (ii) by targeting differentially the C_{H}1, VH, CL, and VL domains.

Most notably, the in silico findings aligned well with the dynamic results obtained using human polyclonal Fab containing both κ and λ subtypes. Specifically, peptide FRWNFHRNTFFP binds both Fab-κ and Fab-λ by targeting the C_{H}1 and C_{L} domains with high affinity (K_{D,in silico}˜5.3·10⁻⁷-1.2·10⁻⁶ M). The distinction between the K and A subtypes lays in sequence-based and structural differences in the light chain. Peptide IIHRRAFWWFPN-GSG demonstrated preferential targeting of the C_{L} domain-specifically, near the hinge region and at the interface with the V_{L} domain—with moderate affinity (K_{D,in silico}˜4.1-6.0·10⁻⁶ M); conversely, peptide NNWWRHARIINA-GSG targets preferentially their counterparts on Fab-λ (K_{D,in silico}˜3.2-6.4·10⁻⁶ M). The in silico structures did not reveal any preferential binding of IRHHNIWFNTIA-GSG and RWTIWPPNWHPP-GSG to either Fab-κ or Fab-λ, mirroring the broad, yet weak, Fab binding recorded experimentally.

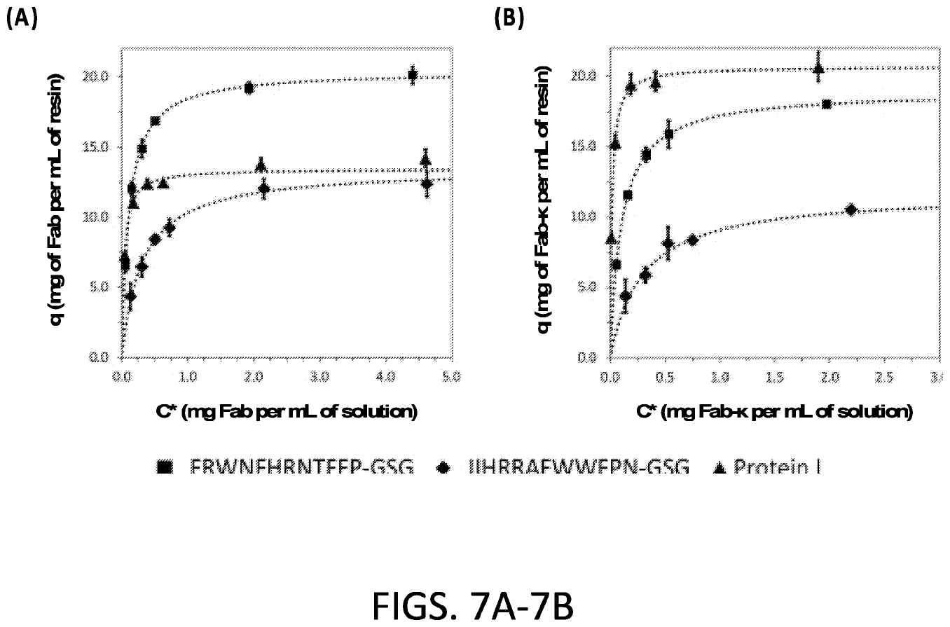

Further validation of the in silico results came from the experimental values of K_{D} derived from the isotherm Fab adsorption studies conducted on peptide-Toyopearl resins. To this end, solutions of human whole Fab and Fab-κ at different titers were incubated with FRWNFHRNTFFP- and IIHRRAFWWFPN-Toyopearl as well as Protein L-agarose resins and the resultant adsorption data were fit against a Langmuir isotherm to derive the values of maximum binding capacity (Qₘₐₓ) and dissociation constant (K_{D,isotherm}) (FIG. 7). The resulting values, collated in Table 7, match rather closely the corresponding in silico results (K_{D,in silico}), confirming the claim of moderate, yet veritably affinity-like, binding strength observed extensively in prior work with peptide ligands.

Table 7. Values of maximum Fab and Fab-κ binding capacity (Qₘₐₓ) and K_{D,isotherm} obtained by fitting the adsorption in FIG. 7 against a Langmuir isotherm.

| | Fab | | Fab-κ | |
|---|---|---|---|---|
| | | Qₘₐₓ (mg | | Qₘₐₓ (mg |
| | K_{D, isotherm} | per mL | K_{D, isotherm} | per mL |
| Resin | (M) | of resin) | (M) | of resin) |
| FRWNFHRNTFFP- | 2.16 · 10⁻⁶ | 20.41 | 2.05 · 10⁻⁶ | 18.96 |
| Toyopearl | | | | |
| IIHRRAFWWFPN- | 5.14 · 10⁻⁶ | 13.48 | 5.82 · 10⁻⁶ | 11.92 |
| Toyopearl | | | | |
| Protein L-Sepharose | 7.10 · 10⁻⁸ | 13.45 | 3.09 · 10⁻⁸ | 20.72 |

Notably, the K_{D} and Qₘₐₓ of FRWNFHRNTFFP and IIHRRAFWWFPN for Fab and Fab-κ are nearly identical, corroborating the universal biorecognition activity of these peptide ligands for all Fab isotypes; as anticipated, Protein L shows a comparably strong binding for Fab-K, but a substantially lower effective capacity for human polyclonal Fab, which comprises ˜30-40% of the Fab-λ isotype, which this ligand does not target. The values of binding capacity of FRWNFHRNTFFP and Protein L fluctuate between 19 and 21 mg of Fab per mL of resin, confirming the effectiveness of the peptide-based adsorbent as alternative to Protein L for Fab purification.

### Example 4

Correlating the conductivity and pH of the binding buffer to product purity. The purification performance of affinity adsorbents is affected significantly by the conductivity and pH of the both the feedstock and the buffers utilized for resin equilibration and wash. In protein ligands, the structure and physicochemical features of the binding domain are determined by the framework of the surrounding tertiary structure; specifically, the network of non-covalent and (when present) disulfide bonds reduce the impact of variations in composition and pH of the aqueous environment. Conversely, the target-binding segment of small peptide ligands comprises the majority or totality of their secondary structure. As a result, the dependence of the binding affinity and selectivity of peptide ligands on the binding environment is often more pronounced than that of their protein counterparts. This poses the need for a careful optimization of the chromatographic protocol, in particular focusing on binding and washing conditions.

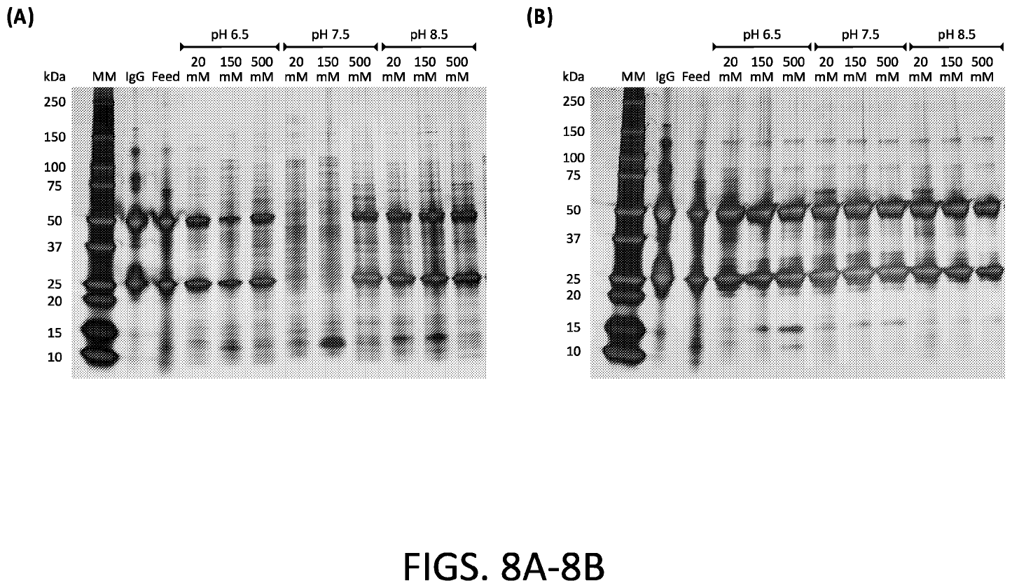

To evaluate these aspects, FRWNFHRNTFFP-Toyopearl resin was elected as the exemplary adsorbent for Fab purification owing to its ability to bind all Fab isotypes and designed a matrix of nine binding buffers constructed using three values of NaCl concentration (20 mM, 150 mM, and 500 mM) and three values of pH (6.5, 7.5, and 8.5). An industrial Chinese hamster ovary (CHO) cell culture fluid containing intact, therapeutic monoclonal Fab with a k light chain at the global titer of 0.72 mg/mL and host cell proteins (HCPs) at the global titer of 0.157 mg/mL was utilized as model feedstock. As anticipated, the Fab binding capacity and selectivity varied substantially amongst the various binding environments and peaked in 50 mM phosphate buffer at pH 7.5 and moderate conductivity—namely, 20 mM or 150 mM NaCl: these conditions are in line with those implemented for ligand selection—namely, PBS at pH 7.4—and afforded full recovery of purified Fab (FIG. 8). Increasing the conductivity of the binding buffer improved the purity of the eluted Fab, but also caused a significant reduction of yield. Therefore, 20 mM NaCl in 50 mM phosphate buffer at pH 7.5 was chosen as the binding buffer for all subsequent applications of the FRWNFHRNTFFP-Toyopearl resin. Notably, by documenting the binding and elution of the whole Fab-κ and its light chain, the electrophoretic analysis confirmed the ability of FRWNFHRNTFFP to target both the heavy and the light chain of Fab.

### Example 5

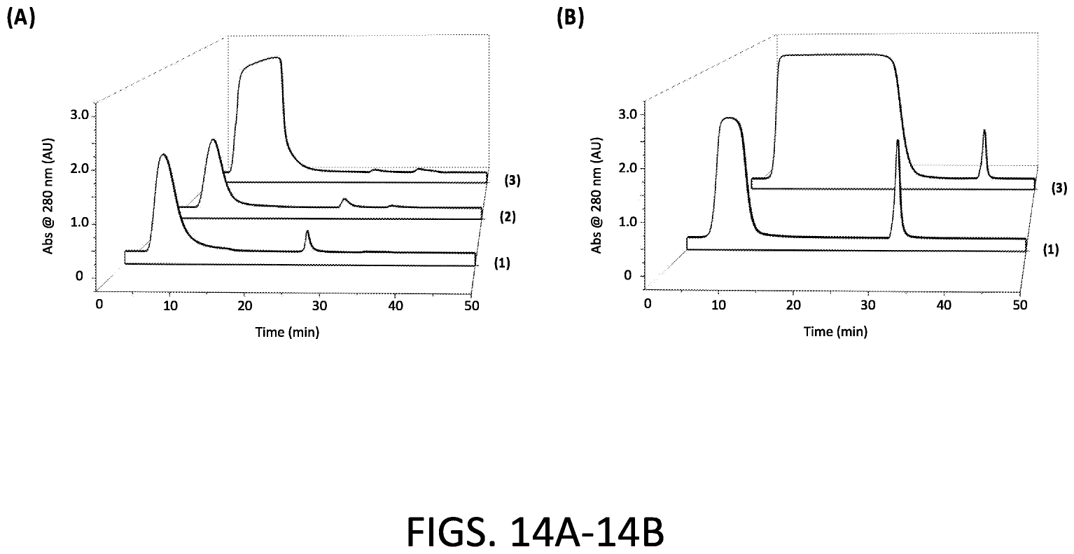

Purification of human Fab from model and industrial recombinant sources. Following the optimization of the adsorption and washing conditions, the FRWNFHRNTFFP-Toyopearl resin was evaluated by purifying human Fabs from three model feedstocks. These included two mimetics of industrial fluids, prepared by spiking either human Fab or Certolizumab in a clarified cell culture fluid (CCCF) produced using non-expressing (“null”) CHO-S cells to a final titer of 1 mg of Fab and 0.135 mg of CHO HCPs per mL, and 1 mg of Certolizumab and 0.109 mg of CHO HCPs per mL, respectively. Certolizumab, commercialized under the name of Cimzia, is a PEG-ylated monoclonal Fab′ antibody domain that targets TNF-α and has been developed for treating rheumatoid arthritis and Crohn's disease. The third fluid was a CHO-K1 industrial harvest featuring a CHO HCP titer of 0.157 mg/mL and containing a therapeutic Fab-κ antibody at 0.171 mg/mL (note: the target antigen was not disclosed). The chromatograms resulting from the purification of Fab from the above-listed feedstocks using FRWNFHRNTFFP-Toyopearl resin are collated in FIG. 14. The chromatographic fractions were analyzed by anti-human Fab and anti-CHO HCP ELISA assays, size-exclusion chromatography (SEC), and SDS-PAGE to calculate the values of product recovery and purity (Table 8).

Table 8. Values of Fab yield, global purity, and removal of HCPs (LRV) obtained by processing three recombinant feedstocks using FRWNFHRNTFFP-Toyopearl resin and Protein L resin. Fab yield was measured via anti-human Fab ELISA, global purity was measured via SEC analysis, and HCP LRV was measured via anti-CHO HCP ELISA.

| | FRWNFHRNTFFP-Toyopearl | | | | | |
|---|---|---|---|---|---|---|
| | resin | | | Protein L-agarose resin | | |
| | | | HCP | | | HCP |
| Feedstock | Yield | Purity | LRV ^{†} | Yield | Purity | LRV ^{†} |
| Human Fab in null CHO-S CCCF | 94.1% | 94.5% * | 1.97 | 53.7% | 94.1% * | 2.11 |
| Certolizumab in null CHO-S | 96.0% | 96.3% * | 1.88 | + | | |
| CCCF | | | | | | |
| Engineered Fab in CHO-K1 | 93.3% | 88.7% ^{§} | 1.79 | 91.0% | 92.2% * | 2.01 |
| CCCF | | | | | | |

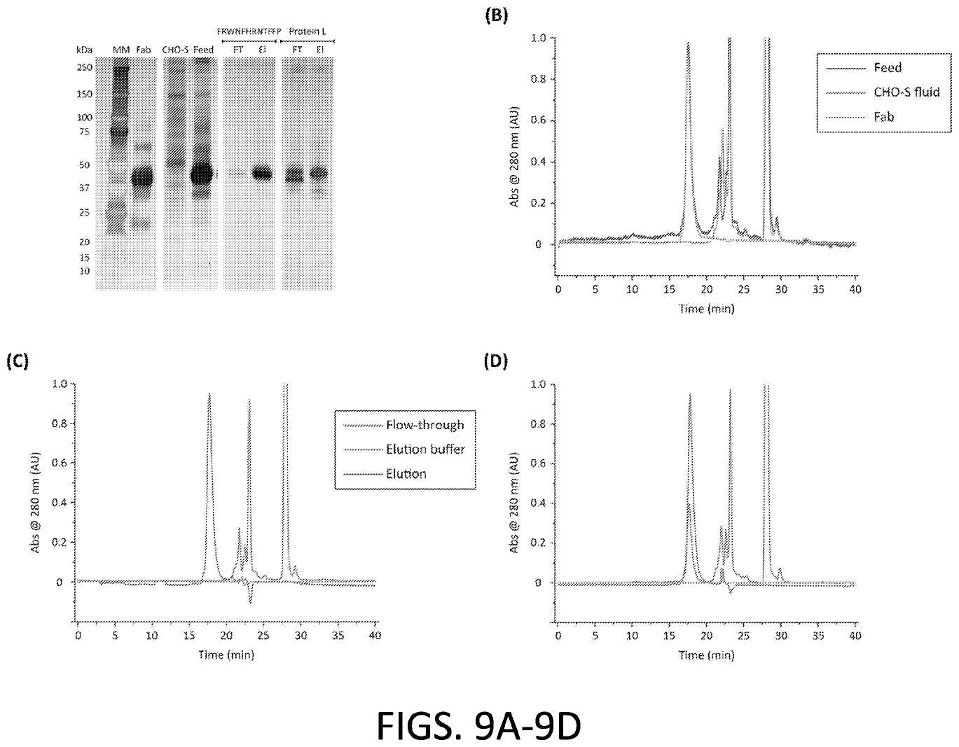

The results reported in FIGS. 9-11 demonstrate the ability of FRWNFHRNTFFP to isolate Fab from complex feedstocks. First, the purification of human polyclonal Fab from a CHO-S fluid resulted in a 94.1% product yield (FIGS. 9A and 9C), a 93-fold reduction of HCPs (corresponding to an HCP LRV˜1.97), and a 135-fold reduction of host cell DNA (corresponding to a DNA LRV˜2.13). Protein L-agarose resin slightly outperformed the peptide-based adsorbent in terms of impurity clearance, affording an HCP LRV˜2.11 and a DNA LRV˜2.71; its product yield, on the other hand, was far lower (53.7%; FIGS. 9A and 9D), likely due to the flow-through of Fab-κ2 and Fab-λ. While satisfactory, the values of HCP and DNA clearance are somewhat lower than those offered by Protein A resins for the purification of full mAbs: while FRWNFHRNTFFP and Protein L may not match engineered Protein A ligands in binding selectivity, these results may also be partly attributed to the association of HCPs to the Fab product. As noticed in a prior study, a significant number of CHO-S HCPs can bind human polyclonal Fab, thus appearing as contaminants in the eluted fraction.

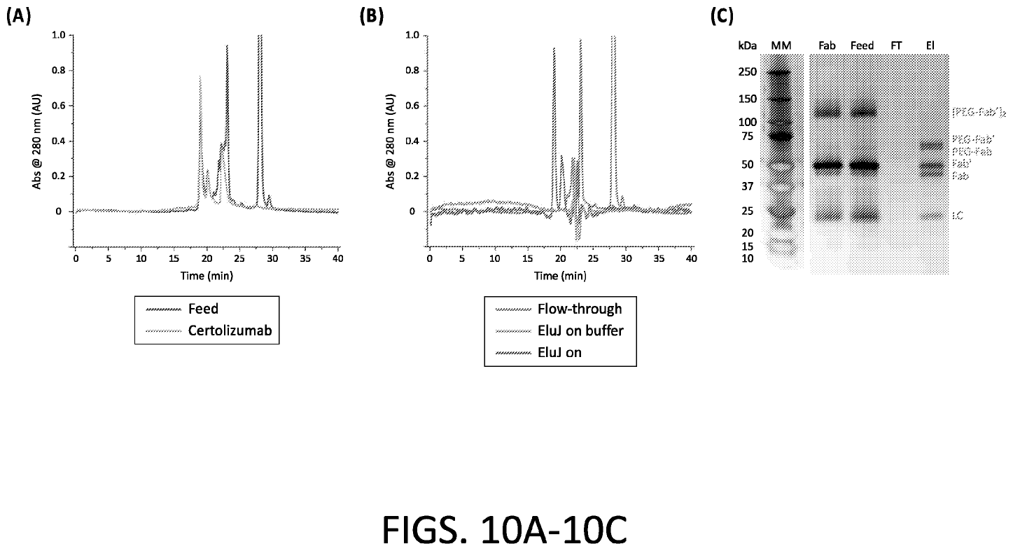

Analogous results were obtained by purifying Certolizumab pegol from the same CHO-S cell culture fluid (FIG. 10). The sample characterization revealed the presence of several species: (i) the PEG-ylated Fab′, which the SDS-PAGE analysis presents as a physical dimer (˜125 kDa) in the feedstock and a monomer (˜63 kDa) in the eluted fraction (note 1: the reported molecular weight is ˜87.8 kDa; note 2: the SEC analysis reported the monomer only); (ii) the free Fab′ (˜50 kDa) and the light chain of Fab (˜25 kDa); and (iii) two minor species, visible only in the SDS-PAGE analysis, which may represent the PEG-ylated and free Fab (note: the molecular weight difference between Fab′ and Fab is ˜3 kDa). The analysis of the flow-through and elution fractions demonstrated that Certolizumab was captured completely and eluted from the FRWNFHRNTFFP-Toyopearl resin with high yield (96.5%) and purity (HCP LRV˜1.88 and a DNA LRV˜2.44).

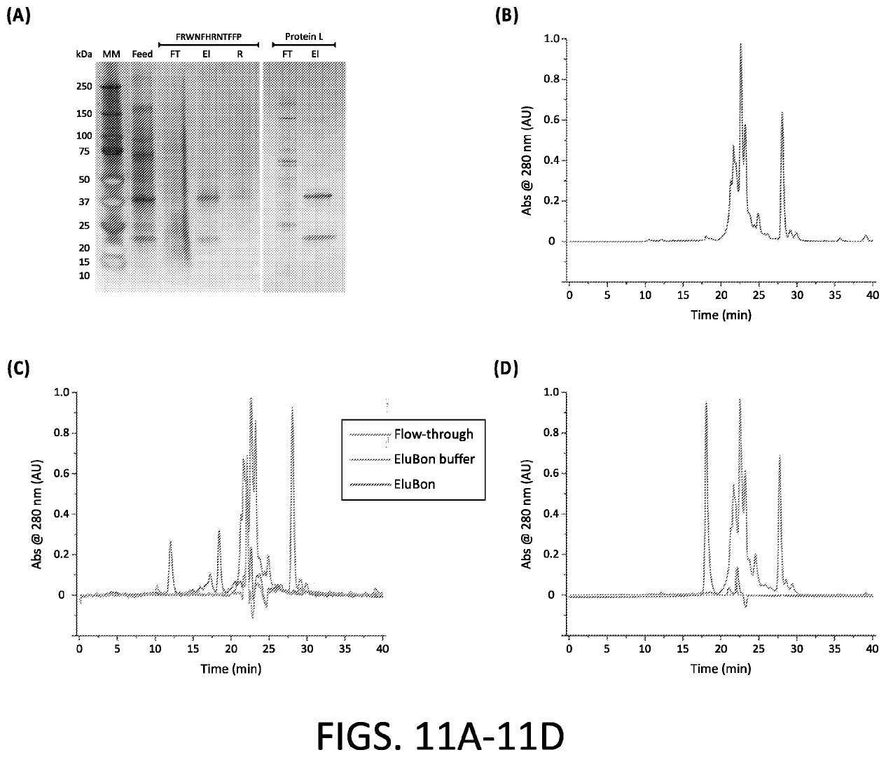

Purification was attempted of an engineered monoclonal Fab from a clarified CHO-K1 cell culture fluid. As shown in FIG. 11A, both the heavy and light chains of this Fab feature a lower molecular weight than their conventional counterparts. It is not uncommon for monoclonal Fabs to present different molecular weights; for example, Kirley et al. and Karageorgos et al. reported the SDS-PAGE analysis of two Fabs that displayed bands at ˜42 and 50 kDa. Unlike the two case studies discussed above, this third Fab purification effort focused on a more challenging feedstock, featuring a lower Fab titer (0.171 mg/mL) and a higher HCP concentration (0.157 mg/mL). Nonetheless, the peptide ligand demonstrated excellent binding affinity, recovering Fab with high yield (93.3%) and purity. The SEC analysis of the samples demonstrate that the Fab is enriched 8-fold from a complex feedstock to a rather pure eluted fraction for both FRWNFHRNTFFP-Toyopearl and Protein L-agarose. However, while a single peak for Fab is registered in the SEC analysis of the Protein L eluate (FIG. 11D), two peaks are found in the sample eluted from the peptide-based adsorbent (FIG. 11C); a species with higher molecular weight can be observed in the corresponding SDS-PAGE analysis in FIG. 11A. At the same time, the HCP and DNA quantification—via ELISA and PicoGreen assays, respectively—exclude the attribution of that peak to CHO-derived impurities: the HCP and DNA LRVs, in fact, ranged between 1.79-2.01 and 2.44-2.96, respectively (Table 8). Accordingly, it was speculated that the higher molecular weight, product-related species exists—or is triggered—in the elution at pH 4 from FRWNFHRNTFFP-Toyopearl resin, and does not exist—or is dissociated—by the harsh elution conditions (pH 2.5) utilized with Protein L-agarose resin.

### Example 6

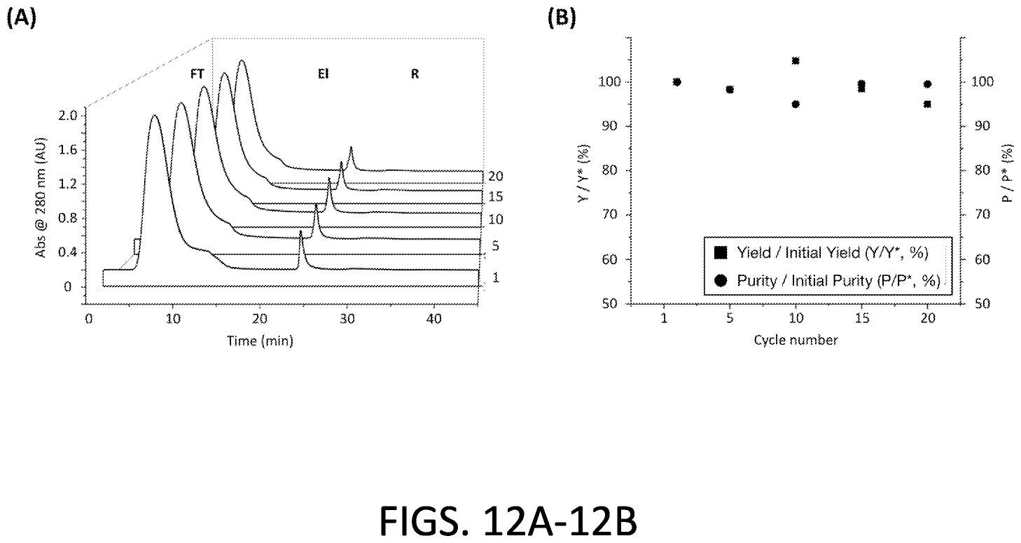

Lifetime study of FRWNFHRNTFFP-Toyopearl resin. The reusability of an affinity resin for product capture is a key requirement for its adoption in an industrial mAb purification pipeline. Currently, Protein A- and Protein L-based resins on the market warrant between 150 and 200 cycles of reuse with intermediate caustic cleaning, without significant loss in binding capacity. In a previous study, a method granting a strong chemical stability to peptide-Toyopearl adsorbents was developed for affinity purification of human antibodies. This method was implemented in this study to produce a stable FRWNFHRNTFFP-Toyopearl resin that can withstand multiple cycles of chromatographic purification of Fab, each followed by harsh cleaning and regeneration in place (i.e., 2 M urea and pH 2.5). The values of Fab yield and purity obtained across 20 cycles of injection of a CHO-S CCCF containing human Fab at 1 mg/mL demonstrate the stability of FRWNFHRNTFFP-Toyopearl resin (FIG. 12). A minor shift was observed in peak shape from sharp (1^{st} cycle) to slightly fronting (20^{th} cycle), although that did not translate into a loss of product yield and quality. Together with the purification case studies discussed above, this lifetime study demonstrates the feasibility of the proposed peptide ligand as alternative to protein ligands for the purification of Fab and Fab-derived therapeutics.

### Example 7

The affinity adsorbent FRWNFHRNTFFP-Toyopearl resin was utilized to purify full-length polyclonal IgG from a Pichia pastoris (P. pastoris) harvest. The resin features a dynamic binding capacity (at 10% breakthrough, DBC_{10%}) of 13.43 mg IgG per mL resin at a 2 minute residence time, along with a yield of 33% and a logarithmic reduction value of HCPs (HCP LRV) of 1.27. Notably, no IgG was detected in the flow-through, as indicated by analytical size exclusion chromatography (SEC) and Protein G chromatography (PrG HPLC). For comparison, Protein A resin provided a yield of 58% (with no IgG detected in the flow-through), while LigaTrap Human IgG resin provided a similar yield of 37% (with no IgG detected in the flow-through). Thus, the peptide ligand FRWNFHRNTFFP performs comparably to commercial alternatives. Additionally, FRWNFHRNTFFP-Toyopearl successfully bound and eluted scFv from P. pastoris perfusate.

Purification of IgG from P. pastoris perfusate. The production of therapeutic glycoproteins in non-mammalian hosts, such as the methylotrophic yeast Pichia pastoris, is gaining traction owing to its lower costs and faster production process. P. pastoris offers several advantages over bacterial expression systems, which are limited in their ability to properly form disulfide bonds and folded proteins, lack of appropriate glycosylation, and do not support post-translational modification. The production of full-length IgG in P. pastoris predominantly remains an upstream challenge, with improper folding and glycosylation posing significant obstacles. In anticipation of future advancements that will enable high-titer, humanized IgG production in P. pastoris, we demonstrated the purification of human polyclonal IgG. In this study, the IgG was purified from a P. pastoris perfusate continuously collected from a bioreactor at the rate of 1.5 vessel volumes per day (VVD) and clarified using a 0.22 μm membrane; the retentate, containing all of the cells, was recirculated back into the bioreactor.

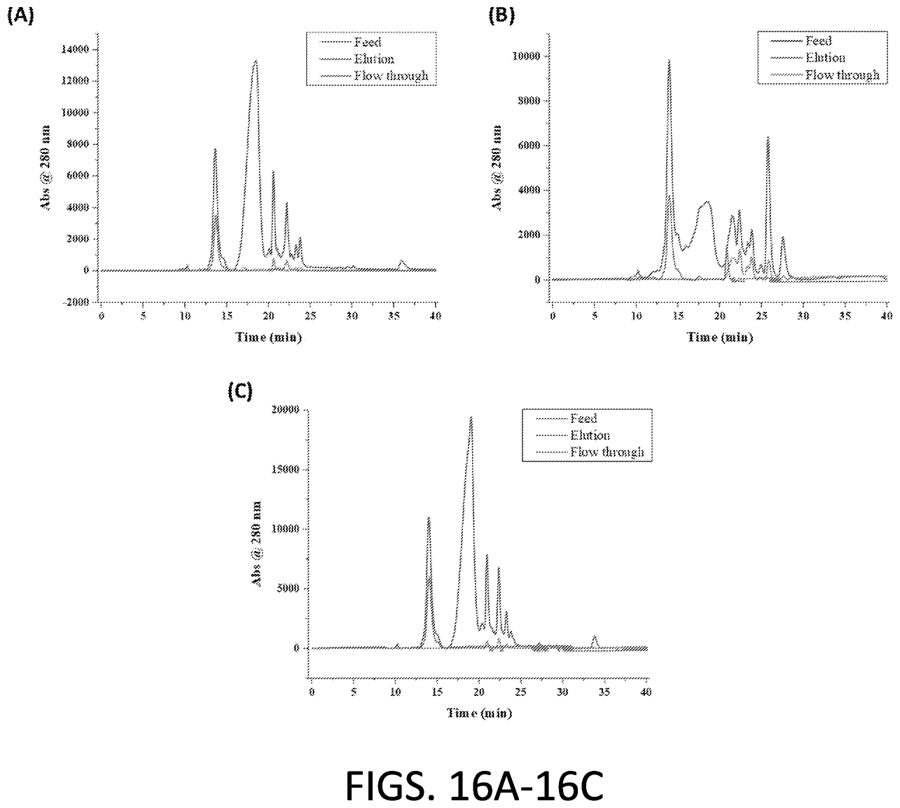

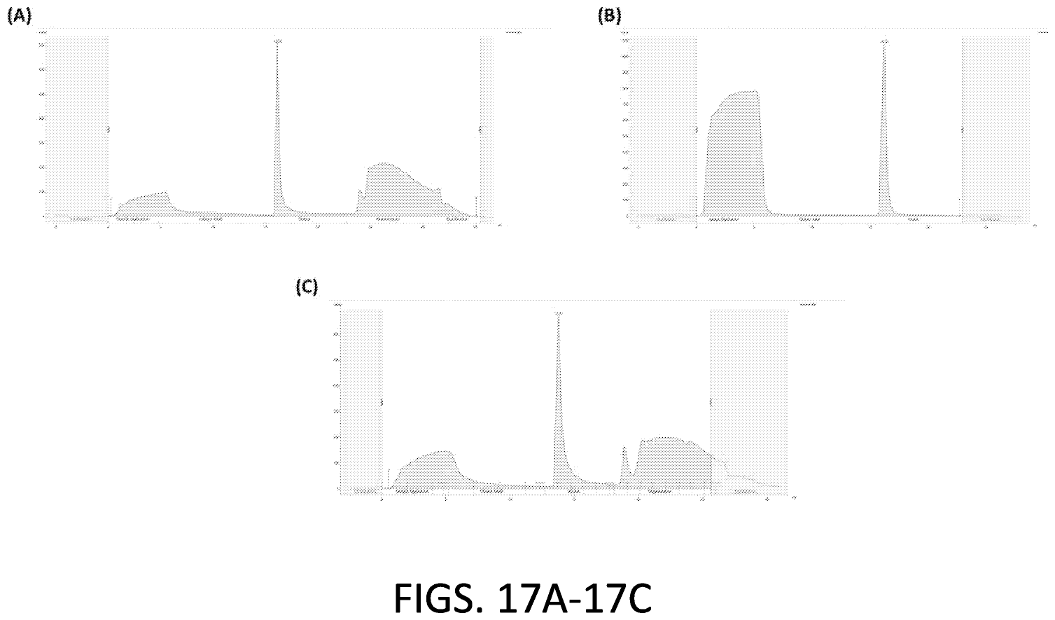

A 0.5 mL column packed with FRWNFHRNTFFP-Toyopearl resin was loaded with 4.8 mL of perfusate. Following resin equilibration and washing with PBS at pH 7.4, the perfusate was loaded at a 2-minute residence time. The bound IgG was eluted with 0.2 M acetate at pH 4, resulting in a 33% yield (Table 9 and chromatograms in FIG. 16A). Despite the low yield, no IgG was detected in the flow-through, as determined by SEC (FIG. 16A) and analytical PrG HPLC. This indicates that all of the loaded IgG was bound, but the majority remained adsorbed on the column during the elution step at pH 4. Protein A resin (MabSelect SuRe LX) was tested under similar conditions and provided a yield of only 58%, with no IgG detected in the flow-through (FIG. 16B; Table 9; FIG. 17B). Another peptide-based affinity adsorbent, commercialized as Ligatrap Human IgG resin, provided a similar 37% yield, with no IgG detected in the flow-through (FIG. 16C; Table 9; FIG. 17C).

The FRWNFHRNTFFP-Toyopearl resin afforded a host cell protein logarithmic reduction value (HCP LRV) of 1.27 as measured via ELISA; SEC analysis of the elution fraction supported this result (FIG. 16A; Table 9). It is not unexpected to observe a lower HCP LRV when purifying proteins from P. pastoris harvests than CHO harvests, given the lower titer of HCPs in the former fluids. Thus, a 1.27 LRV, corresponding to a 94.63% reduction of HCPs in the elution fraction compared to the load, demonstrates the purification activity of the peptide-functionalized resin. Protein A resin (FIG. 16B) and LigaTrap resin afforded similar purity as indicated by the SEC analysis of the eluted fractions (FIG. 16C; Table 9).

Table 9. Values of IgG yield, calculated as a percent ratio of the IgG collected in the elution fraction vs. the loaded IgG, as measured via PrG HPLC analysis of the P pastoris perfusate and elution fractions; values of purity expressed as HCP LRV, calculated as the log₁₀ of the ratio of the mass of HCPs collected in the elution fraction vs. the mass of loaded HCPs, measured via ELISA analysis of the elution fractions and the P pastoris perfusate.

| | | Purity |
|---|---|---|
| Resin | Yield | (HCP LRV) |
| FRWNFHRNTFFP-Toyopearl | 33% | 1.27 |
| MabSelect SuRe LX resin | 58% | — |
| LigaTrap Human IgG resin | 37% | — |

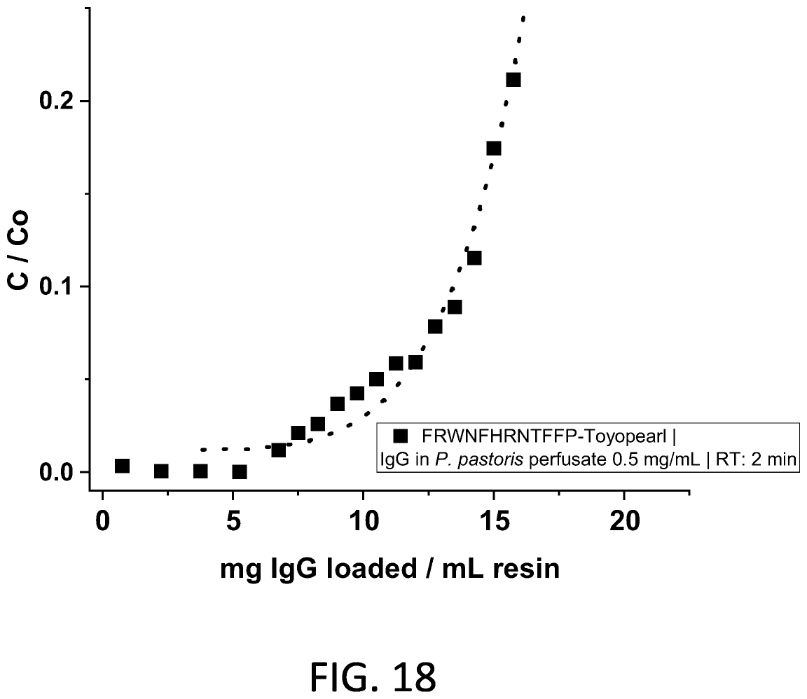

Dynamic IgG binding capacity in competitive conditions (P. pastoris perfusate). The dynamic IgG binding capacity of FRWNFHRNTFFP-Toyopearl at 10% of breakthrough (DBC_{10%}) was measured by loading the clarified P. pastoris perfusate (IgG titer: 0.5 mg/mL) at the residence time (RT) of 2 min. The resulting breakthrough curve is reported in FIG. 18. The DBC_{10%} was found to be 13.4 mg IgG per mL of resin. Previously, where 1 mg Fab/mL was loaded at the RT of 2 min, a DBC_{10%} of 6 mg Fab/mL resin was recorded. In this case, the target (IgG) concentration was reduced to 0.5 mg/mL, which—as anticipated—decreases the measured value of DBC_{10%}.

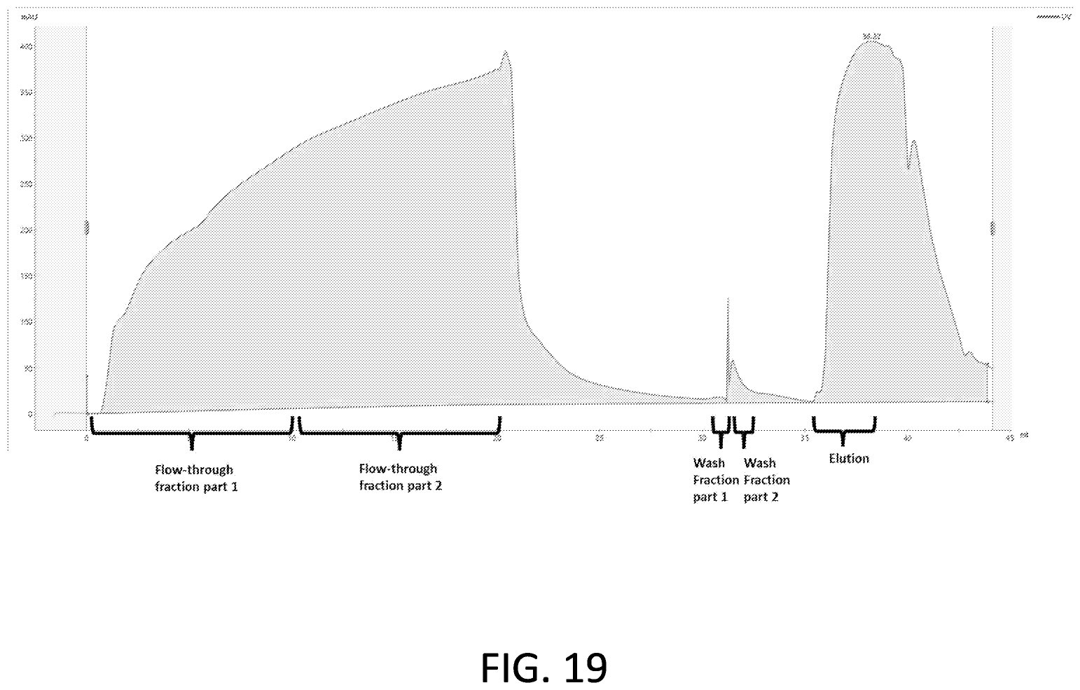

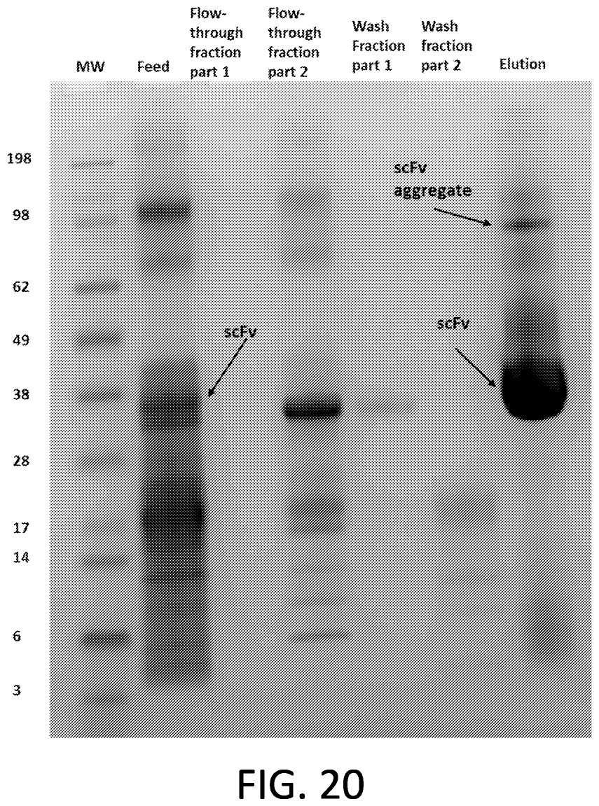

Purification of scFv from a clarified P. pastoris perfusate. A single-chain variable fragment (scFv) is an engineered protein construct that comprises the variable regions of the heavy (V_{H}) and light chains (V_{L}) of the Fab domain of an antibody, connected by a short peptide linker. The purification of these molecules is challenging due to the lack of affinity resins, with most purification being accomplished via affinity tag chromatography (e.g., His-tag). However, the His-tag may sometimes not be effectively accessed by the ligand and must always be cleaved from the product prior to therapeutic use. The peptide ligand FRWNFHRNTFFP was evaluated for the purification of scFv owing to its broad Fab binding activity. The scFv, in fact, comprises segments of the V_{H} and V_{L} regions that present both sequence and structural homology to other segments of Fab. We therefore challenged the FRWNFHRNTFFP-Toyopearl resin against a P. pastoris perfusate containing scFv13R4 at the titer of ˜0.028 mg/mL. A 0.5 mL column packed with FRWNFHRNTFFP-Toyopearl resin was loaded with 20 mL of perfusate at the RT of 3 minutes. The collected fractions (marked based on the chromatogram in FIG. 19) were concentrated and analyzed by SDS-PAGE reported in FIG. 20.

While the molecular weight of monomeric scFv is typically ˜25 kDa, the scFv13R4 presents a molecular weight of ˜36 kDa, due to the added linker and His tag. The SDS-PAGE analysis of the chromatographic fractions obtained by purifying scFv13R4 from the P. pastoris perfusate using FRWNFHRNTFFP-Toyopearl resin returns three key findings: (i) all the scFv molecules loaded until reaching the resin's DBC_{10%} are captured by the peptide ligands; (ii) some of the bound HCPs and other molecules like pigments can be selectively washed out during the pH 4 step as seen in wash fraction part 2; and (iii) scFv is eluted at 0.1 M pH 2.5 at high concentration, indicating that FRWNFHRNTFFP has a higher affinity for scFv than Fab. In the first half of the flow-through, all the scFv loaded to the resin are bound; upon reaching column saturation, the scFv begins to flow-through. As the mobile phase is switched from PBS to 0.2 M acetate pH 4 wash step, a sharp spike is recorded in the chromatogram (FIG. 19—wash fraction part 1). The lane in the SDS-PAGE (FIG. 20) corresponding to wash fraction part 1, which corresponds to the first half of the chromatographic spike, contains scFv at low titer. The second fraction collected during the wash step (Wash fraction part 2) shows no scFv, but an abundance of low molecular weight species, some of which are estimated to be fragmented scFv based on size and known fragmentation patterns. Finally, as the solution of 0.1 M glycine at pH 2.5 is flown through the column, a fraction with high scFv titer is collected. The strong band between 62 kDa and 98 kDa is likely an scFv aggregate as it does not appear in the feed fraction (FIG. 20).

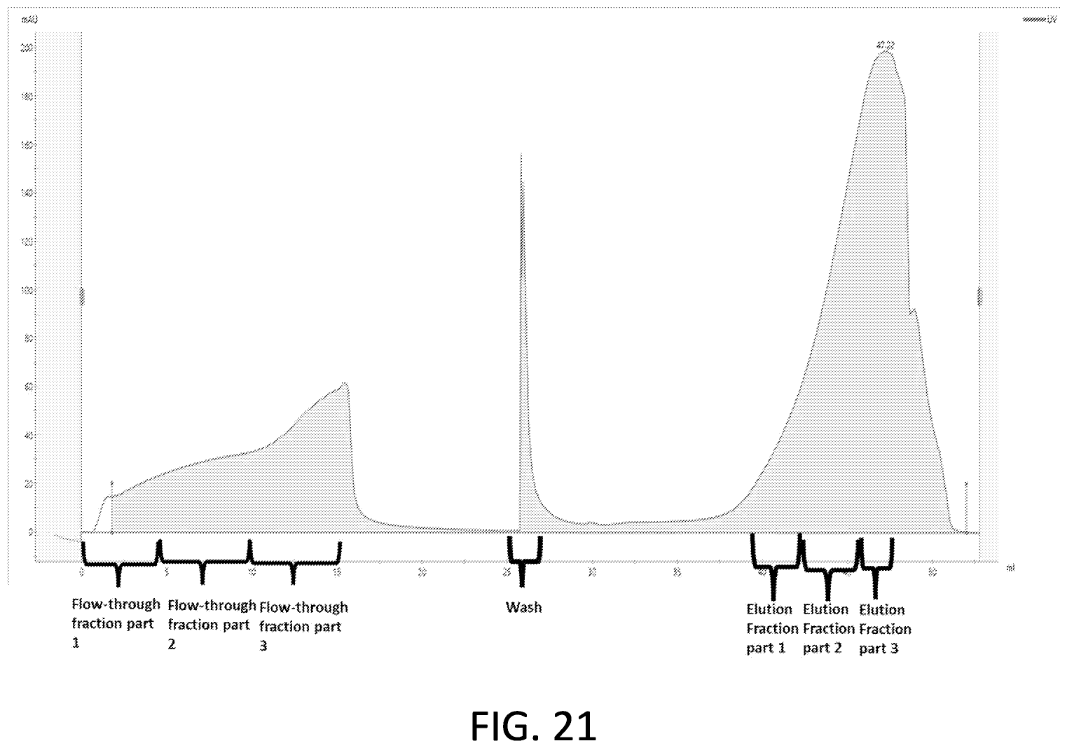

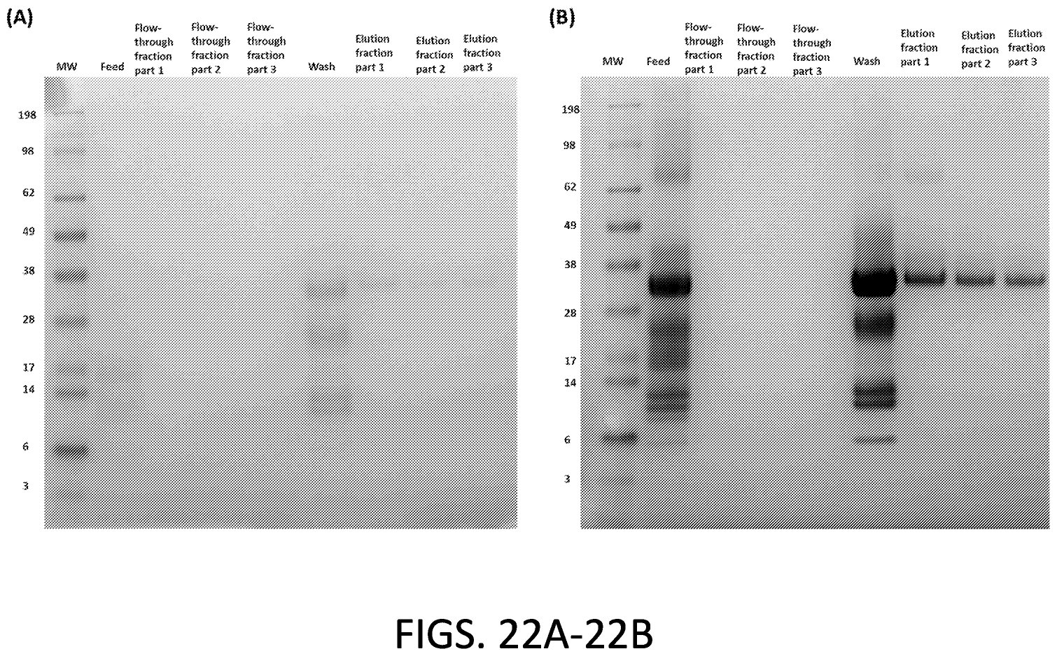

In a subsequent purification effort, dextran-coated charcoal was utilized to strip residual cell culture media as well as the metabolites and pigments secreted by the P. pastoris cells, which can lower the purification performance of chromatographic resins. A 1% w/v suspension was prepared by mixing 1.5 grams of dextran-coated charcoal in 150 mL of the clarified P. pastoris perfusate containing scFv for 5 minutes. After removing the charcoal particles by filtration, 15 mL of the treated perfusate was loaded on a 0.5 mL column packed with FRWNFHRNTFFP-Toyopearl resin at the RT of 3 minutes. The chromatographic fractions were collected as indicated in FIG. 21 and analyzed by SDS-PAGE (FIG. 22): (i) no scFv flowed through; (ii) the purity of the scFv in the eluted fractions is very high.

1. A peptide ligand comprising no more than 12 amino acids and having the amino acid sequence X₁X₂X₃X₄X₅X₆X₇X₈X₉X₁₀X₁₁X₁₂, wherein:
(i) positions X₁-X₅ comprise at least one positively charged amino acid or an aromatic amino acid;
(ii) positions X₆-X₅ comprise at least one aromatic amino acid; and
(iii) positions X₈-X₁₂ comprise at least one aliphatic amino acid or at least one aromatic amino acid;
wherein the peptide ligand is capable of binding a target fragment antigen binding (Fab) domain, or a portion thereof.

2. The peptide ligand of claim 1, wherein the peptide ligand comprises at least one histidine or arginine residue.

3. The peptide ligand of claim 1 or claim 2, wherein the peptide ligand is capable of binding a target Fab comprising a lambda (λ) light chain isotype.

4. The peptide ligand of claim 1 or claim 2, wherein the peptide ligand is capable of binding a target Fab comprising a kappa (κ) light chain isotype.

5. The peptide ligand of claim 1 or claim 2, wherein the peptide ligand is capable of binding a target Fab comprising a lambda (λ) light chain isotype and a target Fab comprising a kappa (κ) light chain isotype.

6. The peptide ligand of any one of claims 1 to 5, wherein the Fab domain and/or the scFv is comprised within a monoclonal antibody or a polyclonal antibody.

7. The peptide ligand of any one of claims 1 to 5, wherein the Fab domain and/or the scFv is comprised within a mono-specific antibody or a multi-specific antibody.

8. The peptide ligand of any one of claims 1 to 5, wherein the Fab domain and/or the scFv is comprised within a single-domain antibody, a humanized antibody, or a chimeric antibody.

9. The peptide ligand of any one of claims 1 to 5, wherein the Fab domain and/or the scFv is comprised within a fusion protein.

10. The peptide ligand of any one of claims 1 to 9, wherein the peptide ligand comprises one or more of alanine (A), glutamic acid (E), phenylalanine (F), histidine (H), isoleucine (I), asparagine (N), proline (P), arginine (R), threonine (T), and tryptophan (W).

11. The peptide ligand of any one of claims 1 to 10, wherein the peptide ligand exhibits a maximum equilibrium binding capacity (Qₘₐₓ) of at least about 15 mg of Fab/mL resin.

12. The peptide ligand of any one of claims 1 to 11, wherein the peptide ligand exhibits a disassociation constant (K_{D}) less than or equal to about 5×10⁻⁶ M.

13. The peptide ligand of any one of claims 1 to 12, wherein the peptide ligand exhibits a dynamic binding capacity (DBC_{10%}) from about 5 mg/mL to about 20 mg/mL.

14. The peptide ligand of any one of claims 1 to 13, wherein the peptide ligand comprises an amino acid sequence having at least 90% sequence identity with any of SEQ ID NOs: 1-19.

15. The peptide ligand of any one of claims 1 to 14, wherein the peptide ligand comprises an amino acid sequence having at least 90% sequence identity with SEQ ID NO: 3.

16. The peptide ligand of any one of claims 1 to 15, wherein the peptide ligand comprises a linker, and wherein the linker is capable of binding to a solid support.

17. The peptide ligand of claim 16, wherein the linker is bound to the C-terminus of the peptide ligand, and wherein the linker comprises a Glyn or a [Gly-Ser-Gly]ₘ, wherein 10≥n≥1 and 6≥m≥1.

18. The peptide ligand of any one of claims 1 to 17, wherein the peptide ligand is bound to a solid support.

19. The peptide ligand of claim 18, wherein the solid support comprises a non-porous or porous particle, a membrane, a polymer surface, a fiber or a woven or non-woven fibermat, a hydrogel, a microplate, and/or a microfluidic device.

20. The peptide ligand of claim 19, wherein the porous particle is a chromatograph resin.

21. The peptide ligand of any one of claims 18 to 20, wherein the solid support comprises polyacrylate, polyacrylamide, poly-ether, polyolefin, polyester, polysaccharide, iron oxide, silica, titania, and/or zirconia.

22. A composition for purifying a target biologic from a fluid wherein the composition comprises any of the peptide ligands of claims 1 to 21.

23. The composition of claim 22, wherein the fluid is a cell culture fluid.

24. The composition of claim 22 or claim 23, wherein the fluid comprises a supernatant and/or a cellular lysate.

25. The composition of any one of claims 22 to 24, wherein the fluid is derived from CHO cells.

26. The composition of claim 25, wherein the CHO cells are selected from the group consisting of: CHO-DXB11 cells, CHO-K1 cells, CHO-DG44 cells, and CHO-S cells, or any derivatives or variants thereof.

27. The composition of any one of claims 22 to 26, wherein the biological fluid is derived from HEK293 cells.

28. The composition of claim 27, wherein the HEK cells are selected from the group consisting of: HEK293S cells, HEK293T cells, HEK293F cells, HEK293FT cells, HEK293FTM cells, HEK293SG cells, HEK293SGGD cells, HEK293H cells, HEK293E cells, HEK293MSR cells, and HEK293A cells, or any derivatives or variants thereof.

29. The composition of any one of claims 22 to 28, wherein the biological fluid is derived from yeast cells or fungal cells.

30. The composition of claim 29, wherein the yeast cells are Pichia pastoris cells.

31. The composition of any one of claims 22 to 30, wherein the fluid comprises a pH from about 3.0 to about 9.0.

32. The composition of any one of claims 22 to 31, wherein the target biologic is:
(i) a monoclonal antibody or a polyclonal antibody;
(ii) a mono-specific antibody or a multi-specific antibody;
(iii) a single-domain antibody, a humanized antibody, or a chimeric antibody; or
(iv) a Fab domain, a scFv, or fusion proteins comprising Fab or scFv.

33. An adsorbent for purifying a target biologic from a fluid wherein the adsorbent comprises any of the peptide ligands of claims 1 to 21.

34. A method of purifying a target biologic from a fluid, the method comprising:
contacting the fluid with any of the peptide ligands of claims 1 to 21, or the adsorbent of claim 33, under conditions sufficient for the peptide ligands to bind the target biologic; and
eluting the target biologic from the peptide ligands.

35. The method of claim 34, wherein the conditions sufficient for the peptide ligands to bind the target biologic comprise use of a buffer comprising:
(i) 10-100 mM phosphate buffer with 0-1 M NaCl at pH 6.5-8.5; or
(ii) 10-100 mM Tris buffer with 0-1 M NaCl at pH 6.5-8.5.

36. The method of claim 34 or claim 35, wherein the conditions sufficient for the peptide ligands to bind the target biologic comprise use of a buffer comprising a pH ranging from about pH 6.0 to about pH 9.0.

37. The method of any one of claims 34 to 36, wherein eluting the target biologic from the peptide ligands comprises use of a buffer comprising:
(i) 0.1 M glycine pH 2.5-3.6; or
(ii) 0.2 M acetate buffer pH 3.6-5.

38. The method of any one of claims 34 to 37, wherein eluting the target biologic from the peptide ligands comprises use of a buffer comprising a pH ranging from about pH 2.0 to about pH 5.0.

39. The method of any one of claims 34 to 38, wherein the method results in a yield for the target biologic of at least 70%.

40. The method of any one of claims 34 to 39, wherein the method results in a purity for the target biologic of at least 70%.
